SEQUENCE LISTING (49 sequences)

SEQ ID NO: 1 - protein, 314 aa, Pyrococcus furiosus
MGKRVVIALGGNALQQRGQKGSYEEMMDNVRKTARQIAEIIARGYEVVITHGNGPQVGSLLLHMDAGQATYGIPAQPMDVAGAMSQGWIGYMIQQALKNELRKRGMEKKVVTIITQTIVDKNDPAFQNPTKPVGPFYDEETAKRLAREKGWIVKEDSGRGWRRVVPSPDPKGHVEAETIKKLVERGVIVIASGGGGVPVILEDGEIKGVEAVIDKDLAGEKLAEEVNADIFMILTDVNGAALYYGTEKEQWLREVKVEELRKYYEEGHFKAGSMGPKVLAAIRFIEWGGERAIIAHLEKAVEALEGKTGTQVLP

SEQ ID NO: 2 - protein, 314 aa, Pyrococcus horikoshii
MPERVVIALGGNALQQRGQKGTYDEMMENVRKTAKQIAEIIARGYEVVITHGNGPQVGTILLHMDAGQSLHGIPAQPMDVAGAMSQGWIGYMIQQALRNELRKRGIEKEVVTIITQTIVDKKDPAFQNPTKPVGPFYDEKTAKKLAKEKGWVVKEDAGRGWRRVVPSPDPKGHVEAETIRRLVESGIIVIASGGGGVPVIEENGEIKGVEAVIDKDLAGEKLAEEVNADILMILTDVNGAALYYGTEKETWLRNVKVEELEKYYQEGHFKAGSMGPKVLAAIRFIKNGGKRAIIAHLEKAVEALEGKTGTQVTP

SEQ ID NO: 3 - protein, 314 aa, Pyrococcus abyssi
MPERVVIALGGNALQQRGQKGTYEEMMENVKKTAKQIAEIIARGYEVVITHGNGPQVGTILLHMDAGQSLHGIPAQPMDVAGAMSQGWIGYMIQQALRNELRKRGIEREVVTIVTQTIVDKDDPAFENPTKPVGPFYDEETAKKLAKEKGWVVKEDAGRGWRRVVPSPDPKRHVEAETIKKLVEDGVIVIASGGGGVPVIEEDGEIKGVEAVIDKDLAGERLAEEVNADILMILTDVNGAALYYGTEKETWLREVKVDEMERYYQEGHFKAGSMGPKVLAAIRFVKNGGKRAIIAHLEKAVEALEGKTGTQVIP

SEQ ID NO: 4 - protein, 314 aa, Thermococcus sp.
MKRVVIALGGNAILQRGQKGTYEEQMENVRKTARQIADIIERGYEVVITHGNGPQVGALLLHMDVGQQVYGIPAQPMDVAGAMTQGQIGYMIQQALINELRARGIEKPVATIVTQTLVDKNDPAFQNPSKPVGPFYDEETAKKLAREKGWVVVEDSGRGWRRVVPSPDPKGHVEAPVIQDLVEKGFIVIASGGGGVPVVEEDGRLKGVEAVIDKDLAGERLAEEVEADIFMILTDVNGAAINFGKPDERWLGKVTVEELKKYYAEGHFKKGSMGPKVLAVIRFVEWGGERGIIASLDKAVEALEGKTGTQVIKG

SEQ ID NO: 5 - protein, 316 aa, Thermococcus gammatolerans
MILMKRVVIALGGNAILQRGQRGTYEEQMENVRKTAKQIADIIERGYEVVITHGNGPQVGALLLHMDAGQQLYGIPAQPMDVAGAMTQGQIGYMIGQALINELRKRGIDRPVATIVTQTIVDKNDPAFKNPSKPVGPFYDEETAKKLAKEKGWVVIEDAGRGWRRVVPSPDPKGHVEAPVIQDLVEKGFIVIASGGGGVPVIEENGELKGVEAVIDKDLAGEKLAEEVKADIFMILTDVNGAAINFGKPDERWLERVTVEELRKYYEEGHFKRGSMGPKVLAVIRFLEWGGERAIIASLDRAVEALEGKTGTQVFP

SEQ ID NO: 6 - protein, 315 aa, Thermococcus kodakarensis
MKRVVIALGGNAILQRGQKGTYEEQMENVRRTAKQIADIILDGDYEVVITHGNGPQVGALLLHMDAGQQVYGIPAQPMDVAGAMTQGQIGYMIGQALINELRKRGVEKPVATIVTQTIVDKNDPAFQNPSKPVGPFYDEETAKKLAKEKGWTVIEDAGRGWRRVVPSPDPKGHVEAPVIVDLVEKGFIVIASGGGGVPVIEENGELKGVEAVIDKDLAGEKLAEEVKADIFMILTDVNGAAINYGKPDEKWLGKVTVDELKRYYKEGHFKKGSMGPKVLAAIRFVEWGGERAVIASLDRAVEALEGKTGTQVVRE

SEQ ID NO: 7 - protein, 315 aa, Thermococcus onnurineus
MKRVVIALGGNAILQRGQKGTYEEQMTNVMKTAKQIVDIILDGDYEVVITHGNGPQIGALLLHMDAGQQIHGIPAQPMDVAGAMTQGQIGYMIQQAIRNELKRRGVERPVATIVTQTLVDKNDPAFQNPSKPVGPFYDEETAKRLAKEKGWTVIEDSGRGWRRVVPSPDPIGHVETPVIQDLVEKGFIVIASGGGGVPVIEEDGMLKGVEAVIDKDLAGEKLAEEVNADIFMILTDVNGAAINYGKPDEKWLGRVTVEELKRYYNEGHFKKGSMGPKVLAAIRFVEWGGERAVIAALDKAVEALEGKTGTQVIKG

SEQ ID NO: 8 - protein, 316 aa, Thermococcus barophilus
MRKRVVIALGGNAILQRGQKGTYDEQMENVKKTAKQIVDIILNNDYEVVITHGNGPQVGALLLHMDAGQQLYGIPAQPMDVAGAMTQGQIGYMIQQAITNELKRRGIYKPVATIVTQVLVDKNDPAFQNPSKPVGPFYDEETAKRLAKEKEWVVVEDAGRGWRRVVPSPDPKDIIEKDIIRDLVEKGFIVIASGGGGIPVIEENGQLKGVEAVIDKDLAGEKLAEVVNADIFMILTDVNGAAINYGKPNERWLHKVAVDELRKYYEEGHFKKGSMGPKVLAAIRFVEWGGERAVIAALDKAVDALEGRTGTQVIKM

SEQ ID NO: 9 - protein, 315 aa, Thermococcus sibiricus
MRKRVVIALGGNAILQRGQKGTYEEQMENVRKTARQIVDIILDNEYEVVITHGNGPQVGALLLQQDAGEHVHGIPAQPMDVCGAMSQGQIGYMIQQAIMNELRRRGVERPVATIVTQTIVDKNDPAFQHPSKPVGPFYSEETAKKLAKEKGWVVIEDAGRGWRRVVPSPDPKGHVEAPIIQDLVEKEFIVISSGGGGIPVVEENGELKGVEAVIDKDLAGERLAEEVNADIFMILTDVNGAAINYGRPNEKWLEKVTLGEIKRYYEEGHFKKGSMGPKVLAAIRFIEWGGERAIIAALDKAVEALEGKTGTQITR

SEQ ID NO: 10 - DNA, 942 bp, Artificial Sequence
  Codon optimized Pfu CK open reading frame
atgggtaaacgtgttgttattgccctgggtggtaatgcactgcagcagcgtggtcagaaaggtagctatgaagaaatgatggataatgtgcgtaaaaccgcacgtcagattgcagaaatcattgcccgtggttatgaagttgttattacccatggtaatggtccgcaggttggtagcctgctgctgcacatggatgcaggtcaggcaacctatggtattccggcacagccgatggatgttgccggtgcaatgagccagggttggattggttatatgattcagcaggccctgaaaaatgaactgcgtaaacgtggcatggaaaaaaaagtggtgaccattattacccagaccattgtggataaaaatgatccggcatttcagaatccgactaaaccggttggtccgttttatgatgaagaaaccgcaaaacgtctggcacgtgaaaaaggttggattgtgaaagaagatagcggtcgcggttggcgtcgtgttgttccgtcaccggatccgaaaggtcatgttgaagccgaaaccattaagaaactggtggaacgtggtgttattgttattgcaagcggtggtggtggtgttccggttattctggaagatggcgaaatcaaaggtgttgaagccgtgattgataaagatctggcaggcgaaaaactggcagaagaagtgaacgccgatatctttatgattctgaccgatgttaatggtgcagcactgtattatggcaccgaaaaagaacagtggctgcgtgaagttaaagttgaagaactgcgcaaatactatgaagaaggccattttaaagcaggtagcatgggtccgaaagttctggcagccattcgttttattgaatggggtggtgaacgtgcaattattgcccatctggaaaaagcagttgaagcactggaaggtaaaaccggcacccaggttctgccg

SEQ ID NO: 11 - DNA, 945 bp, Pyrococcus furiosus
atgggtaagagggtagtgattgcacttggaggtaacgctcttcagcagcgaggtcaaaagggaagttatgaggagatgatggataacgttcgcaagaccgctaggcaaattgccgaaattatagcgagagggtatgaagttgttatcactcatggaaatggtcctcaagttggaagtcttctccttcatatggatgctgggcaggcaacttatggaattcccgcccaaccaatggatgtggctggtgcaatgagtcagggatggattggttatatgattcagcaggctttgaagaacgagctgaggaagaggggcatggaaaagaaagttgttacaataattacccaaacaattgttgataagaatgatccagcattccaaaaccccacaaagccagtggggccattttacgatgaagagactgcaaaaaggttagccagagaaaagggatggatagttaaagaggattctggtagaggttggagaagagtagttccttctccagatcccaaagggcacgttgaggcagagacgattaaaaagttagtggaaaggggagttatagttattgcaagtggtggaggaggagttcctgtaatacttgaagacggggaaataaagggtgttgaggccgtcatcgacaaggatctggcaggagaaaagcttgctgaggaagtaaatgctgacatattcatgattcttacagatgttaacggcgctgcattatactatggaacggaaaaggagcagtggttaagggaagttaaggtcgaagagcttaggaagtactatgaagagggccactttaaggcgggaagcatggggccaaaggttctagcggctataaggttcatcgagtggggtggagagagggccataatagctcacctagaaaaggcagttgaggctctcgaagggaagactggtactcaagttctcccttaa

SEQ ID NO: 12 - DNA, 945 bp, Artificial Sequence
  Codon optimized nucleotide sequence encoding Pyrococcus horikoshii carbamate kinase
atgccggaacgtgttgttattgcactgggtggtaatgcactgcagcagcgtggtcagaaaggcacctatgatgaaatgatggaaaatgttcgtaaaaccgccaaacaaatcgccgaaattattgcacgtggttatgaagttgttatcacccatggtaatggtccgcaggttggcaccattctgctgcatatggatgcaggtcagagcctgcatggtattccggcacagccgatggatgttgccggtgcaatgagccagggttggattggttatatgattcagcaggcactgcgtaatgaactgcgtaaacgtggtattgaaaaagaagtggtgaccattattacccagaccatcgtggataaaaaagatccggcatttcagaatccgaccaaaccggtgggtccgttttatgatgagaaaaccgcaaaaaaactggccaaagaaaaaggttgggtggttaaagaagatgccggtcgcggttggcgtcgtgttgttccgagtccggatccgaaaggtcatgttgaagcagaaaccattcgtcgtctggttgaaagcggtattattgtgattgcaagcggtggtggtggcgttccggttattgaagaaaatggtgaaatcaaaggtgtggaagccgtgattgataaagatctggcaggcgagaaactggcagaagaagttaacgcagatattctgatgattctgaccgatgttaatggtgcagcactgtattatggcaccgaaaaagaaacctggctgcgcaatgttaaagttgaggaactggaaaaatactaccaagagggtcattttaaagcaggtagcatgggtccgaaagttctggcagcaattcgttttatcaaaaatggtggtaaacgtgccattatcgcccatctggaaaaagcagttgaagcactggaaggtaaaaccggcacccaggttaccccgtaa

SEQ ID NO: 13 - DNA, 945 bp, Pyrococcus horikoshii
atgcccgagagagttgttatcgccctgggaggaaacgcactccagcagagagggcaaaaagggacttatgatgagatgatggagaacgtaaggaaaacggctaaacagatagctgagataattgcgaggggttacgaagtcgttataactcacggtaacggacctcaggttgggacgattctccttcatatggatgcgggacaatcccttcatggaataccagcccaacccatggatgttgccggggcgatgagccaaggatggataggttacatgattcaacaagcgttgaggaatgaactcaggaaaagagggatagaaaaggaagttgtcactataataactcaaacgatagttgacaaaaaagatccagcatttcaaaatccaacgaagcctgtaggcccattctatgatgaaaaaactgcaaagaaacttgcaaaggagaaaggatgggttgtcaaggaagatgcaggaaggggatggagaagggttgtcccaagcccagatcccaagggacacgtcgaggctgaaacgataagaagactcgtcgaaagtgggattatagttatagcaagtgggggaggaggagtccccgtaattgaagagaatggagaaataaaaggcgttgaagccgtgatagacaaagatctagctggcgaaaaattggccgaggaggttaatgcagatatacttatgatactaacggatgtcaacggggccgccctatactatggaacggagaaggaaacttggcttagaaacgttaaggtggaagaactggagaagtactaccaagaggggcactttaaggctggaagcatgggtcctaaagttcttgcagcaataaggttcattaaaaatggaggaaaaagagcaataatagctcaccttgagaaagctgttgaagcccttgaaggaaagacaggaacccaggtgactccctaa

SEQ ID NO: 14 - DNA, 945 bp, Artificial Sequence
  Codon optimized nucleotide sequence encoding Pyrococcus abyssi carbamate kinase
atgccggaacgtgttgttattgcactgggtggtaatgcactgcagcagcgtggtcagaaaggcacctatgaagaaatgatggaaaacgtgaaaaaaaccgccaaacaaatcgccgaaattattgcacgtggttatgaagttgttatcacccatggtaatggtccgcaggttggcaccattctgctgcatatggatgcaggtcagagcctgcatggtattccggcacagccgatggatgttgccggtgcaatgagccagggttggattggttatatgattcagcaggcactgcgtaatgaactgcgtaaacgtggtattgaacgtgaagttgtgaccattgttacccagaccattgtggataaagatgatccggcatttgaaaatccgaccaaaccggtgggtccgttttatgatgaagaaaccgcaaaaaaactggccaaagaaaaaggttgggtggttaaagaagatgccggtcgcggttggcgtcgtgttgttccgagtccggatccgaaacgtcatgttgaagcagaaaccatcaaaaaactggttgaagatggcgttattgttatcgcaagcggtggtggtggcgttccggttattgaagaggatggtgaaatcaaaggtgttgaagccgtgattgataaagatctggcaggcgaacgtctggcagaagaagttaacgcagatattctgatgattctgaccgatgttaatggtgcagcactgtattatggcaccgaaaaagaaacctggctgcgtgaagttaaagtggatgaaatggaacgctattatcaagagggtcattttaaagcaggtagcatgggtccgaaagttctggcagcaattcgttttgttaaaaatggtggtaaacgtgccattatcgcccatctggaaaaagcagttgaagcactggaaggtaaaaccggcacccaggttattccgtaa

SEQ ID NO: 15 - DNA, 945 bp, Pyrococcus abyssi
atgccggagagagtcgtcatagccctcggaggaaacgcccttcagcagaggggtcaaaagggaacgtacgaggagatgatggagaacgttaagaaaactgcaaagcagatagctgaaattatcgcgaggggttacgaagtggttataacccacggaaatggccctcaggtagggacgatcctccttcacatggacgctggccaatcacttcacggaattccagctcagcccatggacgttgctggggcaatgagccagggatggataggttacatgatacagcaggcactgaggaacgagcttaggaagagggggatagagagggaggtagttacgatagttacccagacaatagtagataaggacgatcctgccttcgagaacccaactaaaccagttggcccgttctacgacgaggaaactgcaaagaaactagctaaggaaaagggatgggtagtgaaggaagacgctggaaggggatggaggagggtagttccaagcccagatccaaagaggcacgtagaggctgagaccataaagaagttagttgaggatggcgttatagttatagcgagcggcgggggaggagtgccggtaatagaggaggatggagagataaaaggggtcgaagcggtaatagacaaggatctagcgggagagaggttagctgaggaggttaacgctgacatactgatgatcttaacggatgtaaacggggcagcactctactacggaaccgagaaggagacctggcttagagaagttaaggtggacgagatggagaggtactatcaagaagggcacttcaaggccggaagcatggggccaaaggttttagctgctataaggttcgtgaagaacggaggaaagagggcaataattgctcaccttgagaaagctgttgaagccctggaaggaaagaccgggacacaggttattccctga

SEQ ID NO: 16 - DNA, 948 bp, Artificial Sequence
  Codon optimized nucleotide sequence encoding Thermococcus sp. carbamate kinase
atgaaacgtgttgttattgccctgggtggtaatgcaattctgcagcgtggtcagaaaggcacctatgaagaacaaatggccaatgttatgaaaaccgccaaacaaatcgtggatattattctggatggcgattatgaagtggtgattacccatggtaatggtccgcaggttggtgcactgctgctgcatatggatgcaggtcaggcaacccatggcattccggcacagccgatggatgttgccggtgcaatgacccagggtcagattggttacatgattcagcaggcaattcgcaatgaactgaaacgtcgtggtattgatcgtccggttgccaccattgttacccagaccattgttgataaaaacgatccggcatttcagcatccgagcaaaccggttggtccgttttatgatgaagaaaccgcaaaaaaactggccgaagaaaaaggttgggttgttgttgaagatagcggtcgtggttggcgtcgtgttgttccgagtccggatccgattggtcatgttgaagcagaaattattcaggatctggtggaaaaaggctttattgttattaccagcggtggtggcggtgttccggttattgaagaggatggtaaactgcgtggtgttgaagccgttattgataaagatctggcaggcgaacgtctggcagaagaagttaaagcagatatctttatgatcctgaccgatgttaatggtgcagccgttaattttggtaaaccggatgaacgttggctgggtaaagttgcagttgaagaactgcgtaaatactatgaagagggccatttcaaaaaaggtagcatgggtccgaaagttctggcagcaattcgttttgttgaatggggtggtgaacgtgcagttattgcagcactggatcgtgccgttgaagcactggaaggtaaaaccggcacccaggttattaaaggttaa

SEQ ID NO: 17 - DNA, 948 bp, Thermococcus sp.; identical to SEQ ID NO: 16

SEQ ID NO: 18 - DNA, 951 bp, Artificial Sequence
  Codon optimized nucleotide sequence encoding Thermococcus gammatolerans carbamate kinase
atgatcctgatgaaacgtgttgttattgcactgggtggtaatgcaattctgcagcgtggtcagcgtggcacctatgaagaacaaatggaaaatgttcgtaaaaccgccaaacaaatcgccgatattattgaacgtggttatgaagtggttatcacccatggtaatggtccgcaggttggtgcactgctgctgcatatggatgcaggtcagcagctgtatggtattccggcacagccgatggatgttgccggtgcaatgacccagggtcagattggttacatgattggtcaggcactgattaatgaactgcgtaaacgtggtattgatcgtccggttgccaccattgttacccagaccattgttgataaaaatgatccggcattcaaaaacccgagcaaaccggttggtccgttttatgatgaagaaaccgcaaaaaaactggccaaagaaaaaggttgggtggttattgaagatgccggtcgtggttggcgtcgtgttgttccgagtccggatccgaaaggtcatgttgaagcaccggttattcaggatctggttgaaaaaggctttattgtgattgcaagcggtggtggtggcgttccggtgattgaagaaaatggtgaactgaaaggtgttgaagccgtgattgataaagatctggcaggcgagaaactggcagaagaagttaaagcagatatctttatgattctgaccgatgttaatggcgcagcgattaactttggtaaaccggatgaacgttggctggaacgtgttaccgttgaagaactgcgcaaatattacgaagagggtcattttaaacgcggtagcatgggtccgaaagttctggcagttattcgttttctggaatggggtggtgaacgtgcaattattgcaagcctggatcgtgcagttgaagccctggaaggtaaaaccggcacccaggtttttccgtaa

SEQ ID NO: 19 - DNA, 951 bp, Thermococcus gammatolerans
atgatactaatgaagagggtcgtcatagcccttggcggtaacgcaatcctccagcgaggtcaaaggggaacctacgaggagcagatggagaacgtgagaaagacggcgaagcagatagctgacataatcgagaggggctacgaggtcgttataactcacggaaacggtccccaggttggcgcactactcctccacatggacgcgggccagcagctctacggcataccggcccagccgatggacgtggccggtgcgatgacgcagggacagatcgggtacatgatagggcaggccctaatcaatgagctcagaaaacgcggtatagacaggccagttgcgacgatagtaacccagacgattgtggacaagaatgaccccgcctttaagaacccgagcaagcccgtcgggcccttctacgacgaggagacggcgaagaagctggccaaggagaagggctgggtggtcatcgaggatgcgggaaggggctggaggcgtgttgttccgagtcccgatcccaagggccacgttgaagccccggtgatccaggatctcgtggagaagggcttcatagtcatcgcgagcggtggcggtggcgttcccgtcatagaggagaatggagagcttaaaggcgtcgaggctgtcatagacaaggatttggcgggggaaaagctggccgaggaggtcaaggcggatatcttcatgattctcaccgacgtcaacggtgccgcgataaacttcgggaagccggacgagaggtggcttgagagggtcaccgtcgaggagctcaggaagtactacgaagagggacacttcaagaggggcagcatgggtccgaaggttctggccgtcataagattccttgagtggggcggtgaaagggcgataatagcctcgctcgacagggccgttgaagccctcgaagggaagacgggaacgcaggtttttccctga

SEQ ID NO: 20 - DNA, 948 bp, Artificial Sequence
  Codon optimized nucleotide sequence encoding Thermococcus kodakarensis carbamate kinase
atgaaacgtgttgttattgccctgggtggtaatgcaattctgcagcgtggtcagaaaggcacctatgaagaacaaatggaaaatgttcgtcgtaccgcaaaacaaattgccgatattattctggatggcgattatgaagttgtgattacccatggtaatggtccgcaggttggtgcactgctgctgcatatggatgcaggtcagcaggtttatggtattccggcacagccgatggatgttgccggtgcaatgacccagggtcagattggttacatgattggtcaggcactgattaatgaactgcgtaaacgtggtgttgaaaaaccggttgccaccattgttacccagaccattgttgataaaaacgatccggcatttcagaatccgagcaaaccggtgggtccgttttatgatgaagaaaccgcaaaaaaactggccaaagaaaaaggttggaccgttattgaagatgccggtcgtggttggcgtcgtgttgttccgagtccggatccgaaaggtcatgttgaagcaccggttattgttgatctggttgaaaaaggctttattgtgattgcaagcggtggtggtggcgttccggtgattgaagaaaatggtgaactgaaaggtgttgaagccgtgattgataaagatctggcaggcgagaaactggcagaagaagttaaagcagatatcttcatgattctgaccgatgttaatggtgcagcgattaactatggtaaaccggatgaaaaatggctgggcaaagttaccgttgatgagctgaaacgttattacaaagaaggccacttcaaaaaaggtagcatgggtccgaaagttctggcagcaattcgttttgttgaatggggtggtgaacgtgcagttattgcaagcctggatcgtgcagttgaagccctggaaggtaaaaccggcacccaggttgttcgtgaataa

SEQ ID NO: 21 - DNA, 948 bp, Thermococcus kodakarensis
atgaagagagttgtcatagccttgggcgggaacgccatactccagcgaggccagaaggggacttacgaggagcagatggagaacgtgaggaggactgccaagcagatcgctgacataatccttgacggcgattatgaagtcgttatcacccacggaaacggtccacaggttggtgcgctccttctccacatggacgctggccagcaggtctacggcatcccagcccagcctatggacgtcgctggagcgatgacccaggggcagataggctacatgatcggccaggcgctcataaacgagcttaggaagagaggggtcgagaagcccgtcgcaacgatagtaacccagaccatcgttgacaagaacgacccagccttccagaacccgagcaagccggtcggcccgttctacgacgaggagactgccaagaagctagcaaaggaaaagggctggacggttatagaagacgccgggaggggctggaggagggtagttccgagtcccgacccgaaggggcacgtagaggctccggtcatagttgacctcgtcgagaagggcttcatcgtcatagcgagcggcggcggtggcgtcccagtgattgaggagaatggagagcttaagggcgttgaggcggttatagacaaagatttagccggtgaaaagctggctgaagaggttaaagcggatatcttcatgatactcacggacgtgaacggcgcggcaataaactacggaaagcccgatgagaagtggctcgggaaggtaaccgtggacgagctgaagcgctattacaaagagggccacttcaagaagggaagcatggggccgaaggttcttgccgcgataaggttcgttgagtggggaggcgagagggcagtaatagcttcccttgacagggccgtcgaggcgcttgaggggaagacggggacgcaggttgtacgggagtga

SEQ ID NO: 22 - DNA, 948 bp, Artificial Sequence
  Codon optimized nucleotide sequence encoding Thermococcus onnurineus carbamate kinase
atgaaacgtgttgttattgccctgggtggtaatgcaattctgcagcgtggtcagaaaggcacctatgaagaacaaatgaccaatgttatgaaaaccgccaaacaaatcgtggatattattctggatggcgattatgaagtggtgattacccatggtaatggtccgcagattggtgcactgctgctgcatatggatgcaggtcagcagattcatggtattccggcacagccgatggatgttgccggtgcaatgacccagggtcagattggttacatgattcagcaggcaattcgcaatgaactgaaacgtcgtggtgttgaacgtccggttgccaccattgttacccagaccctggttgataaaaatgatccggcatttcagaatccgagcaaaccggttggtccgttttatgatgaagaaaccgcaaaacgtctggccaaagaaaaaggttggaccgttattgaagatagcggtcgtggttggcgtcgtgttgttccgagtccggatccgattggtcatgttgaaacaccggttattcaggatctggttgaaaaaggctttattgtgattgcaagcggtggtggcggtgttccggtgattgaagaggatggtatgctgaaaggtgttgaagcggttattgataaagatctggcaggcgaaaaactggcagaagaagttaacgcagatatctttatgattctgaccgatgttaatggcgcagcgattaactatggtaaaccggatgaaaaatggctgggtcgtgttaccgttgaagaactgaaacgctattacaatgaaggccacttcaaaaaaggtagcatgggtccgaaagttctggcagcaattcgttttgttgaatggggtggtgaacgtgcagttattgcagcactggataaagcagttgaagcactggaaggtaaaaccggcacccaggttattaaaggttaa

SEQ ID NO: 23 - DNA, 948 bp, Thermococcus onnurineus
atgaagagggtagttatagctctcggcgggaacgcgattcttcagcgaggtcaaaagggcacttacgaggaacagatgacgaacgtcatgaagaccgcaaagcaaatcgtcgatataatcctcgatggcgattatgaggttgtaatcacccatggaaacggcccccagattggtgcccttctcctccatatggatgctggacagcagattcacggtatcccagcccagcccatggacgttgccggagcgatgactcagggccagataggatacatgatccagcaggcgataagaaacgagctaaagaggaggggcgtggagaggcccgtcgccaccatagtcacccagacgctcgttgacaaaaacgacccggctttccagaacccgagcaagccagtcggtccgttctacgatgaggagacggcaaagaggcttgcaaaggagaagggctggacggtcatcgaagattccggaaggggctggaggcgcgttgtgccgagtccggacccgataggtcacgtcgagacccctgtgattcaggatctagttgagaagggattcatagtcatagccagcggcggcggtggcgttcccgttatcgaggaggatggaatgctcaagggagttgaggccgtcatagacaaagaccttgccggagagaagctcgcagaagaggtaaacgccgacatcttcatgattctaaccgacgtgaacggtgcggcgataaactacggaaagcccgacgagaagtggctcggaagagttaccgtcgaagagctcaagcgctactataatgagggccacttcaagaagggcagcatggggccaaaggttctcgccgctatacgcttcgtcgagtggggcggcgagagggcggttatagccgcgctggataaggccgtggaagcgcttgaaggcaaaaccgggactcaggtcataaagggctga

SEQ ID NO: 24 - DNA, 948 bp, Artificial Sequence
  Codon optimized nucleotide sequence encoding Thermococcus barophilus carbamate kinase
atgcgtaaacgtgttgttattgcactgggtggtaatgcaattctgcagcgtggtcagaaaggcacctatgatgaacaaatggaaaatgtgaaaaaaaccgccaaacaaattgtggatattattctgaataatgattatgaagtggtgattacccatggtaatggtccgcaggttggtgcactgctgctgcacatggatgcaggtcagcagctgtatggtattccggcacagccgatggatgttgccggtgcaatgacccagggtcagattggttacatgattcagcaggcaattaccaatgaactgaaacgtcgcggtatctataaaccggttgcaaccattgttacccaggttctggttgataaaaatgatccggcatttcagaatccgagcaaaccggttggtccgttttatgatgaagaaaccgcaaaacgtctggccaaagaaaaagaatgggttgttgttgaagatgcaggtcgtggttggcgtcgtgttgttccgagtccggatccgaaagatattattgaaaaagatattattcgcgatctggtggaaaaaggctttattgttattgcaagcggtggtggtggtattccggttattgaagaaaatggtcagctgaaaggtgttgaagccgtgattgataaagatctggcaggcgaaaaactggccgaagttgttaatgcagatatctttatgattctgaccgatgtgaatggcgcagccattaactatggtaaaccgaatgaacgttggctgcataaagttgcagttgatgaactgcgcaaatactatgaagaaggccatttcaaaaaaggcagcatgggtccgaaagttctggcagcaattcgttttgttgaatggggtggtgaacgtgcagttattgcagcactggataaagcagttgatgcactggaaggtcgtaccggcacccaggtgattaaaatg

SEQ ID NO: 25 - DNA, 951 bp, Thermococcus barophilus
atgaggaagagggttgttattgccttgggcggaaacgctattcttcagcgtggtcagaaggggacttatgatgagcagatggaaaatgtaaaaaaaactgcaaagcaaattgtcgatataatccttaacaacgattatgaggttgtaattactcatggaaacggtcctcaggttggagccttgcttctccacatggatgctgggcaacaactttatgggattccagctcagccaatggatgttgctggagctatgactcagggtcagataggctacatgatacagcaggctataacaaacgagcttaaacggagggggatttacaagcctgttgcaacaattgtgacacaggttcttgttgacaagaacgatccagcttttcagaatccgtcaaagccagttggaccattctatgatgaggaaacggcaaaaaggctcgccaaagagaaagagtgggttgttgtggaagacgctggtagaggatggcgtagagttgttccatctccggatccaaaggatataattgagaaggacataatcagggatttagttgagaaaggcttcattgtcatagcgtcgggtggtgggggaattccggttatagaggaaaacggacagctcaaaggtgttgaggctgtcattgataaggatctggctggagaaaagctggctgaggttgttaatgctgacatcttcatgattctcactgatgtaaatggggctgcaataaattatggaaagccgaatgaaaggtggctccacaaagttgctgttgatgagctcaggaagtattatgaagaggggcactttaagaaaggcagcatggggcctaaggtcttagcagcgataagatttgtggaatggggcggggagagagcggtcattgctgctcttgataaagcagttgatgcattggaaggcagaactggaacccaagtaatcaaaatgtga

SEQ ID NO: 26 - DNA, 945 bp, Artificial Sequence
  Codon optimized nucleotide sequence encoding Thermococcus sibiricus carbamate kinase
atgcgtaaacgtgttgttattgcactgggtggtaatgcaattctgcagcgtggtcagaaaggcacctatgaagaacaaatggaaaatgttcgtaaaaccgcacgtcagattgtggatattattctggataatgaatatgaagtggtgattacccatggtaatggtccgcaggttggtgcactgctgcttcagcaggatgccggtgaacatgtgcatggtattccggcacagccgatggatgtttgtggtgcaatgagccagggtcagattggttatatgattcagcaggccattatgaatgaactgcgtcgtcgtggtgttgaacgtccggttgcaaccattgttacccagaccattgttgataaaaatgatccggcatttcagcatccgagcaaaccggttggtccgttttatagcgaagaaaccgcaaaaaaactggccaaagaaaaaggttgggtggttattgaagatgcaggtcgtggttggcgtcgtgttgttccgagtccggatccgaaaggtcatgttgaagcaccgattattcaggatctggtggaaaaagaatttattgtgattagcagcggtggtggtggtattccggttgttgaagaaaatggtgaactgaaaggtgttgaagccgtgattgataaagatctggcaggcgaacgtctggccgaagaagttaacgcagatatctttatgattctgaccgatgtgaatggcgcagccattaactatggtcgtccgaatgaaaaatggctggaaaaagttaccctgggcgaaattaaacgctactatgaagaaggccatttcaaaaaaggtagcatgggtccgaaagttctggcagcaattcgttttattgaatggggtggtgaacgtgcaattattgcagcactggataaagcagttgaagcactggaaggtaaaaccggcacccagattacccgt

SEQ ID NO: 27 - DNA, 948 bp, Thermococcus sibiricus
atgagaaagagagttgtcatagctttaggtgggaatgctattcttcaaaggggtcagaaaggtacttatgaagagcaaatggagaatgtgagaaagactgcaaggcagattgttgatattattcttgataatgagtatgaagtggttatcactcatggtaatggtcctcaagttggagctcttcttctccaacaggatgctggagagcacgtgcatggaattcctgctcaacctatggatgtttgtggtgcaatgagtcagggtcaaataggttacatgattcagcaggcaataatgaatgaattgaggaggaggggtgttgaacggccagtggcgactatagtcacccaaactattgtggacaagaatgatcccgcttttcagcacccatcaaaaccagttgggcccttctatagtgaagagactgctaaaaaactcgctaaggagaaaggttgggtggttatagaggacgctggaaggggttggaggagggtagtgccaagtccagacccaaagggacatgtagaggctcccataatccaagatcttgttgaaaaagaatttatagtgatatcttcaggtggtggaggaattcctgttgtagaggagaatggtgagcttaagggcgttgaagcagttattgacaaagatctggctggagaaaggcttgctgaggaagttaacgctgatattttcatgattcttacggatgttaatggagctgcaattaactatggtagacctaacgaaaagtggcttgaaaaggttactttgggagaaataaagaggtattatgaggaaggccacttcaaaaagggtagcatggggccaaaagtacttgcagcaataaggtttattgagtggggtggggagagggcaataatagcggcactggataaggctgttgaggcattggaaggaaaaacaggcacccaaataacaagatga

SEQ ID NO: 28 - protein, 311 aa, Fervidobacterium nodosum
MKKLAVVAIGGNAVNRPGEEPTAENMIKNLSETAHYLAGMLDEYDIIITHGNGPQVGNLLVQQDLAKHVIPPFPIDVNDAQTQGSLGYMIALTLENELKRRNIERQIAAIVTQIEVDKNDPAFQKPTKPVGPFYSKEEAEKLAQEKGWIMKEDAGRGYRRVVPSPIPLDIVEKEVIKMLVEKDVIVIAAGGGGIPVVKENGMFKGVEAVIDKDRASALLAKEVEADILIILTGVEKVCINYKKPDQFEVDKLTVEEAKKYLAEGQFPSGSMGPKIEAAIDFVSSTGRECIITDMAVLDKALKGETGTKIVP

SEQ ID NO: 29 - protein, 312 aa, Thermosipho melanesiensis
MKKLAVVAIGGNAVNRPGEKPTAENMLKNLSETAKYIVNMIDEYDVAITHGNGPQVGNLLVQQEIAKDKIPPFPIDVNDAQTQGSLGYMIAQSIRNQLKAIGKDMEISAVVTQIIVDKNDPAFQNPTKPVGPFYTEEEAKMLEKEKGWKIVEDAGRGWRRVVPSPKPLDIVEKDVIKLLMKNDVMVIAAGGGGIPVIVEDNKLKGVEAVIDKDRASALLAKEVDADILIILTGVEKVYLNFGKEDQKALDKITTTEAKKFLQEGHFPKGSMGPKIEAAIDFVESTGKECLITDMSVLDKALRGETGTRIIKG

SEQ ID NO: 30 - protein, 323 aa, Anaerobaculum hydrogeniformans
MMAEKVVVALGGNAILQSGQKGTFEEQMENVMATARQIVRMLEAGYEVVVTHGNGPQVGAILIQNELGSGLVPSMPMDVCGAESQGMIGYMLCQALRNCMLGKSLNDWQPCCMVTQVEVDPNDPAFAKPTKPVGPFYTAEEAKKRMSGKNEIWIEDSGRGWRRVVPSPDPKKIVEGEVIKHLSDERYLVIACGGGGIPVIKDEDGTYRGVEAVIDKDLAGERLAQEVGADIFMILTDVPKVAINYKKPDEKWLDVVTPEELRAYEAEGHFKAGSMGPKVKAALRFVENGGKRAIIAKLDSALEALEGKTGTQVVPVKEKICCR

SEQ ID NO: 31 - protein, 315 aa, Aminobacterium colombiense
MGKRILVALGGNAILQPGQKGTSAEQVTNIDKTVKQLIDVVEKGYELVLTHGNGPQVGAILIQHEMAKEAIPAMPLDFCGAESQGLIGYMLCQSIRNECAGRGLKKEAVCIVTQVEVDPHDKAFRNPTKPVGPFYTQAEAEQNMAERGERWIEDAGRGWRKVVPSPEPKEIVEKEVIRALVDQGTVVIASGGGGIPVCRNDQGQLYGVEAVIDKDLAGERLALDVGADIFVILTDVSHAILHYNTPQQKPLKKVTLEEMKKYIEEGHFRAGSMGPKVRAALNFVKKGGGRAIIARLDQVIPALKGEVGTQIESTL

SEQ ID NO: 32 - protein, 318 aa, Thermanaerovibrio acidaminovorans
MAATKVVVALGGNALQEAGTPPTAEAQLEVVKKTATYLAEISKRGYEMAVVHGNGPQVGRIVLSQEIAAQANPETPAMPFDVCGAMSQGYIGYQIQQALRDALRNRNLNIPVVTLVTQVVVDANDPAFKNPTKPIGPFYTEEEAKKLMAEKGYVMKEDAGRGWRRVVASPEPKKITEISAVKRLWDTTIVVTAGGGGIPVVENMDGSLSGVAAVIDKDLAAEKLAEEIEADILLILTEVDKVCINFKKPNQQELSHMTVAEAIKYMEEGHFAPGSMLPKVMAAVKFARTFPGKKAIITSLYKAVDALEGREGTVVTMA

SEQ ID NO: 33 - protein, 310 aa, Halothermothrix orenii
MARIVIALGGNALGKTPLEQREAVKNTAQPIVDLIEDGHEIILAHGNGPQVGMINLAFETAAQNDDNVPEMPFPECGAMSQGYIGYHLQNAIRNELVNRGINKSITSVVTQVLVDKNDNAFEHPTKPVGSFYTEDEARKLIEEKGYRMVEDSGRGYRRVVPSPIPVDIVEKEIIKNLVEDGNIVIACGGGGVPVVEDEEGLKGVPAVIDKDFASEKMAEIVNADLLVILTAVEKVAINFGKENEEWLSEMNVELAQQYCDEGHFAPGSMLPKVKAAMKFASSKEGRKALITSLEKAKEGLAGKTGTLVTA

SEQ ID NO: 34 - protein, 312 aa, Kosmotoga olearia
MKKAVVAIGGNALNKPGEKPSAEAMKKNLLGTVKHLADLIEDGYDLVITHGNGPQVGNLLVQQEIAKDTLPPFPIDVNDAMTQGSIGYLITQTLGNELKSRGTERQIACVLTQIIVDRNDPGFENPTKPVGPFYDEETAKKLQAEKGWIMKEDAGRGWRRVVPSPKPLDVVEIEAIKALTGNDFILVAGGGGGIPVVKKDDGTLEGVEAVIDKDRASALLARLLDADLFLILTAVDHAYINFGKPDQKALERISVNEAKKLMDEGHFAKGSMYPKIESAIDFVESTGKEAIITSLEKVKEAIQGKSGTHITK

SEQ ID NO: 35 - protein, 312 aa, Moorella thermoacetica
MERLAVIAIGGNSLIKNKKLISLYDQLATMRETCRNIADMVEKGWNVVITHGNGPQVGFLIRRAELACGELPLIPLEFAVADTQGAIGYMIQQSLMNEFRRRGIKRQAITIVTQVVVDQNDEAFQNPTKPIGSFMTREEAQRHVEEDGWVVVEDAGRGWRRVVPSPEPKAIVEIRAIKDLIEDGYIVICTGGGGIPVVEHDGNLKGVAAVVDKDNASALLANEIKADVLVISTGVNKVAINFGRPDQKELDRLTIAEAEAYMAAGHFPPGNMGPKITALLRYLKNGGKEGIITSPTELVAALEGKSGTRIVL

SEQ ID NO: 36 - DNA, 933 bp, Artificial Sequence
  Codon optimized nucleotide sequence encoding Fervidobacterium nodosum carbamate kinase
atgaaaaaactggccgttgttgccattggtggtaatgcagttaatcgtccgggtgaagaaccgaccgcagaaaatatgattaaaaacctgagcgaaaccgcacattatctggcaggtatgctggatgaatatgatattatcattacccatggcaatggtccgcaggttggtaatctgctggttcagcaggatctggcaaaacatgttattcctccatttccgattgatgttaatgatgcacagacccagggtagcttaggttatatgattgcactgaccctggaaaatgaactgaaacgtcgtaatattgaacgtcagattgcagcaattgtgacccagattgaagtggataaaaatgatccggcatttcagaaaccgaccaaaccggtgggtccgttttatagcaaagaagaagcagaaaaactggcccaagaaaaaggttggattatgaaagaagatgcaggtcgcggttatcgtcgtgttgttccgagcccgattccgctggatattgttgaaaaagaagtgattaaaatgctggttgaaaaagatgttattgtgattgcagccggtggtggtggtattccggttgttaaagaaaatggtatgtttaaaggtgtggaagccgtgattgataaagatcgtgcaagcgcactgctggcaaaagaagttgaagcagatattctgattattctgaccggtgttgaaaaagtgtgcattaattacaaaaaaccggatcagtttgaagttgataaactgaccgttgaagaagccaaaaaatacctggcagaaggtcagtttccgagcggtagcatgggtccgaaaattgaagcagcaattgattttgttagcagcaccggtcgtgaatgcattattaccgatatggcagttctggataaagccctgaaaggtgaaaccggcaccaaaattgttccg

SEQ ID NO: 37 - DNA, 936 bp, Artificial Sequence
  Codon optimized nucleotide sequence encoding Thermosipho melanesiensis carbamate kinase
atgaaaaaactggccgttgttgccattggtggtaatgcagttaatcgtccgggtgaaaaaccgaccgcagaaaatatgctgaaaaatctgagcgaaaccgccaaatatatcgtgaatatgattgatgaatatgatgtggccattacccatggcaatggtccgcaggttggtaatctgctggttcagcaagaaattgccaaagataaaattcctccgtttccgattgatgttaatgatgcacagacccagggtagtctgggttatatgattgcacagagcattcgtaatcagctgaaagccattggtaaagatatggaaattagcgcagttgtgacccagattattgtggataaaaatgatccggcatttcagaatccgaccaaacctgttggtccgttttataccgaagaagaggcaaaaatgctggaaaaagaaaaaggctggaaaattgttgaagatgcaggtcgtggttggcgtcgtgttgttccgagcccgaaaccgctggatattgttgaaaaagatgtgattaaactgctgatgaaaaatgatgttatggtgattgcagccggtggtggtggtattccggttattgtggaagataacaaactgaaaggtgtggaagccgtgattgataaagatcgtgcaagcgcactgctggcaaaagaagttgacgcagatattctgattattctgaccggtgtggaaaaagtgtatctgaattttggcaaagaagatcagaaagccctggataaaatcaccaccaccgaagccaaaaaattcttacaagaaggtcattttccgaaaggtagcatgggtccgaaaattgaagcagccattgattttgttgaaagcaccggtaaagaatgcctgattaccgatatgagcgttctggataaagcactgcgtggtgaaaccggcacccgtattatcaaaggt

SEQ ID NO: 38 - DNA, 972 bp, Artificial Sequence
  Codon optimized nucleotide sequence encoding Anaerobaculum hydrogeniformans carbamate kinase
atgatggccgaaaaagttgttgttgcactgggtggtaatgcaattctgcagagcggtcagaaaggcacctttgaagaacaaatggaaaatgttatggcaaccgcacgtcagattgttcgtatgctggaagcaggttatgaagttgtggttacccatggtaatggtccgcaggttggtgccattctgattcagaacgaactgggtagcggtctggttccgagcatgccgatggatgtttgtggtgcagaaagccagggtatgattggttatatgctgtgtcaggcactgcgtaattgtatgctgggtaaaagcctgaatgattggcagccgtgttgtatggtgacccaggttgaagttgatccgaatgatccggcatttgcaaaaccgaccaaaccggtgggtccgttttataccgcagaagaggcaaaaaaacgtatgagcggcaaaaacgaaatctggattgaagatagtggtcgtggttggcgtcgtgttgttccgagtccggatccgaaaaaaatcgttgaaggtgaagtgatcaaacacctgagtgatgaacgttatctggttattgcatgcggtggtggtggtattccggttattaaagatgaagatggcacctatcgtggtgttgaagcagttattgataaagatctggcaggcgaacgtctggcacaagaagttggcgcagatatctttatgattctgaccgatgttccgaaagtggccatcaattacaaaaaaccggatgagaaatggctggatgttgttacaccggaagaactgcgtgcatatgaagcagaaggtcattttaaagcaggtagcatgggtccgaaagttaaagcagcactgcgttttgttgaaaatggtggtaaacgtgccattatcgcaaaactggatagcgcactggaagccctggaaggtaaaaccggtacacaggttgttccggttaaagaaaaaatctgctgtcgctaa

SEQ ID NO: 39 - DNA, 948 bp, Artificial Sequence
  Codon optimized nucleotide sequence encoding Aminobacterium colombiense carbamate kinase
atgggtaaacgtattctggttgcactgggtggtaatgcaattctgcagcctggtcagaaaggcaccagcgcagaacaggttaccaatattgataaaaccgtgaaacagctgatcgatgtggtggaaaaaggttatgaactggttctgacccatggtaatggtccgcaggttggtgcgattctgattcagcatgaaatggcaaaagaagcaattccggcaatgccgctggatttttgtggtgcagaaagccagggtctgattggttatatgctgtgtcagagcattcgtaatgaatgtgcaggtcgtggtctgaaaaaagaagccgtttgtattgttacccaggttgaagttgatccgcatgataaagcatttcgtaatccgaccaaaccggtgggtccgttttatacccaggccgaagcagaacagaatatggcagaacgtggtgaacgttggattgaagatgccggtcgtggttggcgtaaagttgttccgagtccggaaccgaaagaaatcgttgaaaaagaagttattcgcgcactggttgatcagggcaccgttgttattgcaagcggtggtggtggtattccggtttgtcgtaatgatcagggtcagctgtatggtgttgaagcagtgattgataaagatctggcaggcgaacgtctggcactggatgttggtgcagatatttttgttattctgaccgatgttagccatgccatcctgcattataacacaccgcagcagaaaccgctgaaaaaagttaccctggaagaaatgaaaaaatacatcgaagaaggccattttcgcgcaggtagcatgggtccgaaagttcgtgcagcactgaattttgttaaaaaaggtggtggtcgtgcaattattgcccgtctggatcaggttattccggcactgaaaggtgaagttggcacccagattgaaagcaccctgtaa

SEQ ID NO: 40 - DNA, 957 bp, Artificial Sequence
  Codon optimized nucleotide sequence encoding Thermanaerovibrio acidaminovorans carbamate kinase.
atggcagcaaccaaagttgttgttgcactgggtggtaatgcactgcaagaagcaggcacccctccgaccgcagaagcacagctggaagttgtgaaaaaaaccgcaacctatctggccgaaattagcaaacgtggttatgaaatggcagtggttcatggtaatggtccgcaggttggtcgtattgttctgagccaagaaattgcagcacaggcaaatccggaaacaccggcaatgccgtttgatgtttgtggtgcaatgagccagggttatattggttatcagattcagcaggcactgcgtgatgcactgcgtaatcgtaatctgaatattccggttgttaccctggttacccaggttgttgtggatgcaaatgatccggcattcaaaaatccgaccaaaccgattggtccgttttataccgaagaagaggcaaaaaaactgatggccgaaaaaggctatgtgatgaaagaagatgcaggtcgcggttggcgtcgtgttgttgccagtccggaaccgaaaaaaatcaccgaaatttcagcagttaaacgcctgtgggataccaccattgttgttaccgcaggcggtggtggtattccggtggttgaaaatatggatggtagcctgagcggtgttgcagcagttattgataaagatctggcagcagaaaaactggccgaagaaattgaagccgatattctgctgattctgaccgaagttgataaagtgtgcatcaacttcaaaaaaccgaatcagcaagaactgagccatatgaccgttgccgaagcaatcaaatatatggaagaaggtcattttgcaccgggtagcatgctgccgaaagttatggcagccgttaaatttgcacgtacctttccgggtaaaaaagccattattaccagcctgtataaagcagttgatgccctggaaggtcgtgaaggcaccgttgttaccatggcataa

SEQ ID NO: 41 - DNA, 933 bp, Artificial Sequence
  Codon optimized nucleotide sequence encoding Halothermothrix orenii carbamate kinase
atggcacgtattgttattgcactgggtggtaatgccctgggtaaaacaccgctggaacagcgtgaagcagtgaaaaataccgcacagccgattgttgatctgattgaagatggccatgaaattattctggcacatggtaatggtccgcaggttggtatgattaatctggcatttgaaaccgcagcacagaatgatgataatgttccggaaatgccgtttcctgaatgtggtgcaatgagccagggttatattggttatcatctgcagaatgccattcgcaatgaactggttaatcgcggtattaacaaaagcattaccagcgttgttacccaggttctggttgataaaaatgataacgcatttgagcatccgaccaaaccggttggtagcttttataccgaagatgaagcacgcaaactgatcgaagaaaaaggttatcgtatggtggaagatagcggtcgtggttatcgtcgtgttgttccgagcccgattccggttgatattgttgaaaaagagatcatcaaaaacctggtcgaagatggcaatattgtgattgcgtgcggtggtggtggcgttccggttgttgaggatgaagaaggtctgaaaggcgttcctgcagttattgataaagattttgccagcgaaaaaatggccgaaattgttaatgccgatctgctggttattctgaccgcagtggaaaaagttgcaatcaactttggcaaagaaaacgaagaatggctgagcgaaatgaatgttgaactggcacagcagtattgtgatgaaggtcattttgcaccgggtagcatgctgccgaaagttaaagcagcaatgaaatttgcaagcagcaaagaaggtcgtaaagcactgattaccagcctggaaaaagcaaaagaaggcctggcaggtaaaaccggcaccctggttaccgcataa

SEQ ID NO: 42 - DNA, 939 bp, Artificial Sequence
  Codon optimized nucleotide sequence encoding Kosmotoga olearia carbamate kinase
atgaaaaaagccgttgttgccattggtggtaatgcactgaataaaccgggtgaaaaaccgagcgcagaagcaatgaaaaaaaacctgctgggcaccgttaaacatctggcagatctgattgaagatggttatgatctggttattacccatggtaatggtccgcaggttggtaatctgctggttcagcaagaaattgcaaaagataccctgcctccgtttccgattgatgttaatgatgcaatgacccagggtagcattggttatctgattacccagaccctgggcaatgaactgaaaagccgtggcaccgaacgtcagattgcatgtgttctgacccagattattgtggatcgtaatgatccgggttttgagaatccgaccaaaccggtgggtccgttttatgatgaagaaaccgcaaaaaaactgcaggcagaaaaaggctggatcatgaaagaagatgcaggtcgcggttggcgtcgtgttgttccgagcccgaaaccgctggatgttgttgaaattgaagcaattaaagccctgaccggcaacgattttattctggttgccggtggtggtggcggtattccggttgttaaaaaagatgatggcaccctggaaggtgttgaagcagttattgataaagatcgtgcaagcgcactgctggcacgtctgctggatgcagacctgtttctgattctgaccgcagttgatcatgcctatatcaattttggtaaaccggatcagaaagccctggaacgtattagcgttaatgaagccaaaaaactgatggatgaaggccattttgccaaaggtagcatgtatccgaaaattgaaagcgccattgattttgttgaaagcaccggtaaagaagccattattaccagcctggaaaaagtgaaagaagcgattcagggtaaaagcggcacccatattaccaaataa

SEQ ID NO: 43 - DNA, 939 bp, Artificial Sequence
  Codon optimized nucleotide sequence encoding Moorella thermoacetica carbamate kinase
atggaacgtctggcagttattgcaattggtggtaatagcctgatcaaaaacaaaaaactgatcagcctgtatgatcagctggcaaccatgcgtgaaacctgtcgtaatattgcagatatggttgaaaaaggctggaacgttgttattacccatggtaatggtccgcaggttggttttctgattcgtcgtgcagaactggcatgtggtgaactgccgctgattccgctggaatttgcagttgcagatacccagggtgccattggttatatgattcagcagagcctgatgaatgaatttcgtcgtcgtggtattaaacgtcaggccattacaattgttacccaggttgttgtggatcagaatgatgaagcatttcagaatccgaccaaaccgattggtagctttatgacccgtgaagaagcacagcgtcacgttgaagaagatggttgggttgttgttgaagatgcaggtcgtggttggcgtcgtgttgttccgagtccggaaccgaaagcaattgttgaaattcgtgcaatcaaagacctgatcgaagatggctatattgttatttgtaccggtggtggtggtattccggttgttgaacatgatggtaatctgaaaggtgttgcagccgttgtggataaagataatgcaagcgcactgctggccaatgaaatcaaagcagatgttctggttattagcaccggtgtgaataaagtggcaattaactttggtcgtccggatcagaaagaactggatcgtctgaccattgccgaagccgaagcatatatggcagcaggtcattttccgcctggtaatatgggtccgaaaattacagcactgctgcgctatctgaaaaatggtggtaaagaaggtattattaccagcccgaccgaactggttgcagcactggaaggtaaaagcggcacccgtattgttctgtaa

SEQ ID NO: 44 - protein, 310 aa, Enterococcus faecalis
MGKKMVVALGGNAILSNDASAHAQQQALVQTSAYLVHLIKQGHRLIVSHGNGPQVGNLLLQQQAADSEKNPAMPLDTCVAMTQGSIGYWLSNALNQELNKAGIKKQVATVLTQVVVDPADEAFKNPTKPIGPFLTEAEAKEAMQAGAIFKEDAGRGWRKVVPSPKPIDIHEAETINTLIKNDIITISCGGGGIPVVGQELKGVEAVIDKDFASEKLAELVDADALVILTGVDYVCINYGKPDEKQLTNVTVAELEEYKQAGHFAPGSMLPKIEAAIQFVESQPNKQAIITSLENLGSMSGDEIVGTVVTK

SEQ ID NO: 45 - protein, 315 aa, Clostridium tetani
MDNKTILVALGGNAILQPGQFASYENQLNNVKTSCRVLARLIKKGYRLIITHGNGPQVGNIIRQNEEASSVVPPMPMDVCSAESQGFIGYMIQQSMMNELVNLGLEIPVVTFVTRVEVYEKDSAFKNPTKPIGMFYSEEQAKKLMEEKQWILKPDANRGWRRVVPSPKPINIVETKSIRKLIDEGNIVIACGGGGIPVMRCEDNTYKGVEAVIDKDRSGCKLAQQAEADMFIILTDVENACINYGKENQKSLGKVKIEELEQYIEKGEFSKGSMLPKVESAVEFVKRTNGISIICALDKAELAIEGKAGTIIRKA

SEQ ID NO: 46 - DNA, 930 bp, Artificial Sequence
  Codon optimized nucleotide sequence encoding Enterococcus faecalis carbamate kinase
atgggcaaaaaaatggttgttgcactgggtggtaatgccattctgagcaatgatgcaagcgcacatgcacagcagcaggcactggttcagaccagcgcatatctggttcatctgattaaacagggtcatcgtctgattgttagccatggtaatggtccgcaggttggtaatctgcttctgcaacagcaggctgcagatagcgaaaaaaatccggcaatgccgctggatacctgtgttgcaatgacccagggtagcattggttattggctgagcaatgcactgaatcaagaactgaataaagccggtatcaaaaaacaggttgcaaccgttctgacccaggttgttgttgatccggcagatgaagcattcaaaaatccgaccaaaccgattggtccgtttctgaccgaagccgaagcaaaagaagcaatgcaggcaggcgcaatctttaaagaagatgcaggtcgcggttggcgtaaagttgttccgagcccgaaaccgattgatattcatgaagcagaaaccattaataccctgattaaaaacgatatcattaccattagctgcggtggtggtggtattccggttgttggccaagaactgaaaggcgttgaagcagttattgataaagattttgccagcgaaaaactggccgaactggttgatgcagatgcactggttattctgaccggtgttgattatgtgtgcattaactatggcaaaccggatgaaaaacagctgaccaatgttaccgttgcagaactggaagaatataaacaggcaggtcattttgcaccgggtagcatgctgccgaaaattgaagcagcaattcagtttgttgaaagccagccgaataaacaggcaattattaccagcctggaaaatctgggtagcatgagcggtgatgaaattgttggcaccgttgtgaccaaa

SEQ ID NO: 47 - DNA, 945 bp, Artificial Sequence
  Codon optimized nucleotide sequence encoding Clostridium tetani carbamate kinase
atggataacaaaaccattctggttgccctgggtggtaatgcaattctgcagcctggtcagtttgcaagctatgaaaatcagctgaataatgtgaaaaccagctgtcgtgttctggcacgtctgatcaaaaaaggttatcgcctgattattacccatggtaatggtccgcaggttggtaacattattcgtcagaatgaagaagccagcagcgttgttcctccgatgccgatggatgtttgtagcgcagaaagccagggttttattggttatatgattcagcagagcatgatgaatgaactggttaatctgggtctggaaattccggttgttacctttgttacccgtgttgaagtgtatgaaaaagatagcgccttcaaaaatccgaccaaaccgattggtatgttttatagcgaagaacaggccaaaaaactgatggaagaaaaacagtggattctgaaaccggatgcaaatcgtggttggcgtcgtgttgttccgagcccgaaaccgattaacattgttgaaaccaaaagcattcgcaaactgattgatgaaggcaatattgttattgcctgtggtggtggtggtattccggttatgcgttgtgaagataacacctataaaggtgtggaagccgtgattgataaagatcgtagcggttgtaaactggcacagcaggccgaagcagatatgtttatcattctgaccgatgtggaaaatgcctgcattaactatggcaaagaaaatcagaaaagcctgggcaaagtgaaaattgaagaactggaacagtatattgaaaaaggcgaatttagcaaaggtagcatgctgccgaaagttgaaagcgcagttgaatttgttaaacgcaccaatggcattagcattatttgtgcactggataaagcagaactggccattgaaggtaaagcaggcaccattattcgtaaagcc

SEQ ID NO: 48 - DNA, 32 bp, Artificial Sequence
  primer
gtggtttccatgggtaagagggtagtgattgc

SEQ ID NO: 49 - DNA, 36 bp, Artificial Sequence
  primer
gcattcgctaagctgggtcttctaaagttcctcagg

(19) United States
(12) Patent Application Publication
(10) Pub. No.: US 2014/0377815 A1
(43) Pub. Date: Dec. 25, 2014

(54) Methods of Producing Carbamoyl Phosphate and Urea

(71) Applicants: James Edward Hennessy, Barton (AU); Amy Philbrook, Duffy (AU); Daniel Miles Bartkus, Narrabundah (AU); Christopher John Easton, Barton (AU); Colin Scott, Melba (AU); John G. Oakeshott, Wanniassa (AU); Hye-Kyung Kim, Palmerston (AU); Melissa Jane Latter, O'Connor (AU)

(72) Inventors: James Edward Hennessy, Barton (AU); Amy Philbrook, Duffy (AU); Daniel Miles Bartkus, Narrabundah (AU); Christopher John Easton, Barton (AU); Colin Scott, Melba (AU); John G. Oakeshott, Wanniassa (AU); Hye-Kyung Kim, Palmerston (AU); Melissa Jane Latter, O'Connor (AU)

(21) Appl. No.: 14/125,259

(22) PCT Filed: Jun. 15, 2012
(86) PCT No.: PCT/AU2012/000691

§ 371 Date: Aug. 15, 2014

Related U.S. Application Data
(60) Provisional application No. 61/498,395, filed on Jun. 17, 2011.

(51) Int. Cl. C12N 9/12; C12P 13/10; C02F 3/34; C12P 13/02; C12P 13/00

(52) U.S. Cl. CPC C12N 9/1217; C12P 13/02; C12P 13/00; C02F 3/342; C12P 13/10; C12Y 207/02002
USPC 435/114; 435/129; 435/128; 435/262.5; 536/23.2; 558/153; 564/63

(57) ABSTRACT

The present invention relates to a method of producing carbamoyl phosphate, the method comprising reacting ammonia, ATP, bicarbonate and CO₂, or a hydrated form thereof, in a composition in the presence of a carbamate kinase, wherein the ammonia and CO₂, or hydrated form thereof, are converted to carbamate in a chemical reaction and the carbamate and ATP are converted to carbamoyl phosphate in an enzyme-catalysed reaction by the carbamate kinase, and wherein the pH of the composition is about 8 to about 12. The invention also relates to methods of producing urea.

### FIELD OF THE INVENTION

The present invention relates to a method of producing carbamoyl phosphate, the method comprising reacting ammonia, ATP, bicarbonate and CO₂, or a hydrated form thereof, in a composition in the presence of a carbamate kinase, wherein the ammonia and CO₂, or hydrated form thereof, are converted to carbamate in a chemical reaction and the carbamate and ATP are converted to carbamoyl phosphate in an enzyme-catalysed reaction by the carbamate kinase, and wherein the pH of the composition is about 8 to about 12. The invention also relates to methods of producing urea.

### BACKGROUND OF THE INVENTION

Urea is the most common nitrogen fertiliser and accounts for more than 50% of the world's fertiliser market. This fertiliser is currently manufactured using the energy intensive Bosch-Meiser process from ammonia prepared using the Haber process. The requirement for the large energy and natural gas inputs in urea production has focused urea production to regions that have abundant fossil fuel supplies, as a consequence many countries import significant proportion of their urea and the associated transport costs are high. The high energy input, reliance upon natural gas and high transport costs also couple to the cost of urea fertilisers with the price of fossil fuels, which is sensitive to supply-dependent fluctuations. A further consideration for future impacts on the cost of urea fertilisers is the proposed introduction of the Carbon Pollution Reduction Scheme to be put in place in 2015, because of the associated costs for CO₂ emissions connected with fertiliser production and transport.

Most of the nitrogen applied as urea fertilisers is lost to various waste streams, including animal and municipal wastes. These waste streams contain significant quantities of nitrogen as nitrates, nitrites, ammonia and organic nitrogen compounds (amino acids and nucleotides), which must be removed from the waste stream to prevent eutrophic effects (such as bacterial blooms). The nitrogen in waste water treatments is ultimately lost as gaseous nitrogen oxides (N₂O, a potent GHG, and NO) and nitrogen (N₂). Recycling the nitrogen in these systems would: i) provide a low energy, low GHG source of nitrogen fertiliser; ii) remove nitrogen from waste streams, preventing down-stream eutrophic effects; and iii) reduce the production of nitrous oxides. Additionally, production of urea fertilisers obtained from reclaimed waste nitrogen would likely be distributed locally, reducing the transportation costs and the overall environmental footprint of the product.

The urea cycle is the metabolic process through which nitrogen is appropriately disseminated by a series of five enzymes, detoxifying ammonia to excreted urea in animals and providing nitrogenous metabolic intermediates in other organisms. The first step in the urea cycle is the production of carbamoyl phosphate from carbonic acid, organic phosphorus (ATP), and either ammonia or glutamate, by the enzyme carbamoyl phosphate synthetase.

Ammonia has a pKa of 9.25 and it is therefore ammonium not ammonia that is primarily available at biological pH. In fact 99.4% of ammonia is protonated at pH 7.

The urea cycle is required to transform carbamoyl phosphate to urea due to low levels of ammonia found in most organisms (Jones and Lipman, 1960). Carbamoyl phosphate undergoes another 4 enzymatic transformations finally resulting in the formation of urea. Chemically, these concurrent steps could be eliminated with conversion of carbamoyl phosphate to urea upon treatment with ammonia.

Carbamoyl phosphate is unstable at physiological pH and temperature with a half-life (t_{1/2}) of 5 minutes (Wang et al., 2008). Despite this instability, it undergoes further transformation affording citrulline, arginine, pyrimidine nucleotides and urea. Thermal decomposition of carbamoyl phosphate is avoided through stabilization by transcarbamoylases. Aspartate and ornithine transcarbamoylase reduce the rate of thermal decomposition of carbamoyl phosphate by a factor of 5,000. In solution absent of transcarbamoylases, carbamoyl phosphate decomposes via a planar intermediate (Allen and Jones, 1964). This geometry is prohibited in the active site of aspartate and ornithine transcarbamoylase and the carbamoyl phosphate is thus stabilized and able to be transformed into stable ureido products.

Carbamoyl phosphate is unstable in aqueous environments and readily decomposes (Wang et al., 2008). The pathway through which decomposition occurs is pH dependent. Under acid hydrolysis, decomposition occurs to ammonium, orthophosphate and carbon dioxide (Allen and Jones, 1964), whereas the dianion, present in alkaline conditions, decomposes to orthophosphate and cyanate (Allen and Jones, 1964). The path of decomposition is important for subsequent transformations as further nitrogen substitution is not possible with ammonia and carbonate but is known to occur readily with cyanate (Wen and Brooker, 1994). For example, the formation of urea does not occur when carbonate is treated with ammonia, however it does form when cyanate is treated with ammonia and the production of urea increases with increased ammonia concentration.

The instability of carbamoyl phosphate has deemed it impractical as a commercial intermediate. Thus, there is a need for methods to produce carbamoyl phosphate, as well as methods to produce urea. Furthermore, there is a need for methods for reducing ammonia levels from waste material.

### SUMMARY OF THE INVENTION

The present inventors have developed a method whereby ammonia can be converted to carbamoyl phosphate using a single enzyme.

Thus, in a first aspect the present invention provides a method of producing carbamoyl phosphate, the method comprising reacting ammonia, ATP, bicarbonate and CO₂, or a hydrated form thereof, in a composition in the presence of a carbamate kinase, wherein the ammonia and CO₂, or hydrated form thereof, are converted to carbamate in a chemical reaction and the carbamate and ATP are converted to carbamoyl phosphate in an enzyme-catalysed reaction by the carbamate kinase, and wherein the pH of the composition is about 8 to about 12.

In a preferred embodiment, the pH is about 9.9, about 9 to about 11, about 9.25 to about 11.25, about 10.25 to about 11.25, or about 10.5 to about 11.5. In this embodiment it is preferred that the carbamate kinase is derived from a hyperthermophile bacteria, or is a biologically active mutant thereof. Examples of hyperthermophile bacteria include, but are not limited to, Pyrococcus sp. and Thermococcus sp. Examples of Pyrococcus sp. include, but are not limited to, Pyrococcus abyssi, Pyrococcus endeavori, Pyrococcus glycovorans, Pyrococcus horikoshii and Pyrococcus woesei. Examples of Thermococcus sp. include, but are not limited to, Thermococcus acidaminovorans. Thermococcus aegaeus, Thermococcus aggregans. Thermococcus alcahphilus, Thermococcus atlanticus, Thermococcus barophilus, Thermococcus barossii, Thermococcus celer, Thermococcus celericrescens, Thermococcus chitonophagus, Thermococcus coalescens, Thermococcus fumicolans, Thermococcus gammatolerans, Thermococcus gorgonarius, Thermococcus guaymasensis. Thermococcus hydrothermalis, Thermococcus kodakarensis, Thermococcus litoralis, Thermococcus marinus, Thermococcus mexicalis, Thermococcus nautilus, Thermococcus onnurineus, Thermococcus pacificus, Thermococcus peptonophilus, Thermococcus profundus, Thermococcus radiotolerans, Thermococcus sibiricus, Thermococcus siculi, Thermococcus stetteri, Thermococcus thioreducens, Thermococcus waimanguensis, Thermococcus waiotapuensis and Thermococcus zilligii. Furthermore, examples of such carbamate kinases include those which comprise

a) an amino acid sequence provided as any one of SEQ ID NOs:1 to 9,

b) an amino acid sequence which is at least 50% identical to any one or more of SEQ ID NOs:1 to 9, and/or

c) a biologically active fragment of a) or b).

In another embodiment, the carbamate kinase comprises

a) an amino acid sequence provided as any one of SEQ ID NOs:4 to 9.

b) an amino acid sequence which is at least 50% identical to any one or more of SEQ ID NOs:4 to 9, and/or

c) a biologically active fragment of a) or b).

In a further embodiment, the carbamate kinase comprises

a) an amino acid sequence provided as SEQ ID NOs:1, 8 or 9,

b) an amino acid sequence which is at least 50% identical to any one or more of SEQ ID NOs:1, 8 or 9, and/or

c) a biologically active fragment of a) or b).

In a further embodiment, the carbamate kinase comprises

a) an amino acid sequence provided as SEQ ID NO:8 or SEQ ID NO:9,

b) an amino acid sequence which is at least 50% identical to any one or more of SEQ ID NO:8 or SEQ ID NO:9, and/or

c) a biologically active fragment of a) or b).

In the above embodiments the temperature is about 10° C. to about 100° C. In an embodiment, the temperature is about 20° C. to about 80° C. In another embodiment, the temperature is about 20° C. to about 60° C. In another embodiment, the temperature is about 20° C. to about 30° C.

In another preferred embodiment, at pH 11 0.5 μM of carbamate kinase produces at least 0.5 μmol/min/mg, at least 0.9 μmol/min/mg, or between 0.5 and 3 μmol/min/m, ADP after thirty minutes incubation in NaHCO₃ (0.2 M), ATP (10 mM) and 20 mM NH₄OH at 40° C.

In another preferred embodiment, at pH 11.5 0.5 μM of carbamate kinase produces at least 0.25 μmol/min/mg, at least 0.6 μmol/min/mg, or between 0.25 and 2.5 μmol/min/m, ADP after thirty minutes incubation in NaHCO₃ (0.2 M), ATP (10 mM) and 20 mM NH₄OH at 40° C.

In an alternate embodiment, the pH is about 9 to about 10.5, or about 9.5 to about 10.5. In this embodiment, it is preferred that the carbamate kinase is derived from a thermophile bacteria, or is a mutant thereof. Examples of thermophile bacteria include, but are not limited to, Fervidobacterium sp. (for example, Fervidobacterium nodosum), Thermosipho sp. (for example, Thermosipho melanesiensis), Anaerobaculum sp. (for example, Anaerobaculum hydrogeniformans and Aminobacterium colombiense), Thermanaerovibrio sp. (for example, Thermanaerovibrio acidaminovorans), Halothermothrix sp. (for example, Halothermothrix orenii), Kosmotoga sp. (for example, Kosmotoga olearia) and Moorella sp. (for example, Moorella thermoacetica). Examples of such carbamate kinases include those which comprise

a) an amino acid sequence provided as any one of SEQ ID NOs:28 to 35,

b) an amino acid sequence which is at least 50% identical to any one or more of SEQ ID NOs:28 to 35, and/or

c) a biologically active fragment of a) or b). Furthermore, in an embodiment the temperature is about 10° C. to about 60° C., about 20° C. to about 60° C., about 20° C. to about 40° C., or is about 20° C. to about 30° C. In addition, in an embodiment at pH 10.5 0.5 μM of carbamate kinase produces at least 0.6 μmol/min/mg ADP after thirty minutes incubation in NaHCO₃ (0.2 M), ATP (10 mM) and 200 mM NH₄OH at 40° C.

In a preferred embodiment, the carbamate kinase maintains at least about 50%, at least about 60%, at least about 70%, or at least about 80% of its activity after storage for 1 year at 4° C. and/or storage for 60 hours at 40° C.

In a further embodiment, the pressure is about 0 to about 350 MPa, or between about 1 atm and about 10 atm.

In an embodiment, the method is performed in a continuous system.

In a further embodiment, the carbamate kinase is immobilized on a solid support.

In yet another embodiment, the source of the ammonia is waste material.

In a further embodiment, the method further produces one or both of cyanate and cyanic acid through the decomposition of at least some of the carbamoyl phosphate.

In a further embodiment, the carbamate kinase is a fusion protein comprising at least one other polypeptide. The at least one other polypeptide may be, for example, a polypeptide that enhances the stability of the carbamate kinase, a polypeptide that promotes the secretion of the fusion protein from a cell such as a bacterial cell or a yeast cell, a polypeptide that assists in the purification of the fusion protein and/or a polypeptide that assists in the binding of the polypeptide to a solid support.

The in situ transformation of carbamoyl phosphate to urea and/or other stable transportable commodities enables the replacement of current energy intensive commercial processes with efficient biological pathways. Thus, in a further aspect the present invention provides a method of producing a compound from carbamoyl phosphate, the method comprising

i) performing the method of the invention to produce carbamoyl phosphate, and

ii) performing one or more further reactions to produce the compound.

The present inventors devised a simple procedure for producing urea. Accordingly, in a particularly preferred embodiment the compound is urea and step ii) comprises reacting the carbamoyl phosphate produced from step i) with ammonia to produce urea via an intermediate which is one or both of cyanate and cyanic acid. The ammonia may be that present during step i) and/or additional ammonia added during step ii).

In an embodiment, when producing urea at least step ii) is performed at a temperature of at least about 90° C. More preferably, when producing urea at least step ii) is performed at a temperature of about 90° C. to about 100° C.

In a further embodiment, the method comprises

i) performing the method of the invention to produce carbamoyl phosphate with the carbamate kinase immobilized on a solid support,

ii) separating the carbamoyl phosphate produced by step i) from the solid support,

iii) binding the carbamoyl phosphate to a resin, and

iv) washing the resin in a solution comprising ammonia to convert the carbamoyl phosphate via an intermediate, which is one or both of cyanate and cyanic acid, into urea.

Step iv) not only produces the urea but also liberates the molecule from the resin allowing the urea to be collected in the material eluted from the resin.

In an embodiment, step iv) is performed at a temperature of about 90° C. to about 100° C.

In a further embodiment, the compound is an intermediate of the urea cycle selected from citrulline, argininosuccinate, arginine, ornithine and a combination of two or more thereof.

In another embodiment, the method comprises performing a method of the invention to produce one or both of cyanate and cyanic acid, and reacting the cyanate, and/or cyanic acid with a nucleophile.

In an embodiment, the compound is a pyrimidine. Examples of pyrimidines include, but are not limited to, uracil, cytosine or thymine, or a derivative thereof with one, two or three phosphate groups.

In an embodiment, the method is performed in a single vessel.

In a further aspect, the present invention provides a method of reducing the concentration of ammonia in a waste material, the method comprising performing a method of the invention.

In an embodiment, the waste material is in water.

The present inventors have also identified a polynucleotide which, when expressed in a bacterial cell, results in higher levels of production of a carbamate kinase comprising an amino acid sequence as provided in SEQ ID NO:1 than the native open reading frame (SEQ ID NO:11). Thus, in a further aspect, the present invention provides an isolated and/or exogenous polynucleotide encoding a carbamate kinase, wherein the polynucleotide comprises a sequence of nucleotides provided as SEQ ID NO:10. In another aspect, the present invention provides an isolated and/or exogenous polynucleotide encoding a carbamate kinase, wherein the polynucleotide comprises a sequence of nucleotides provided as any one of SEQ ID NOs:10, 12, 14, 16, 18, 20, 22, 24, 26 or 36 to 43.

In a preferred embodiment, the polynucleotide is operably linked to a promoter capable of directing expression of the polynucleotide in a cell.

Also provided is a vector comprising a polynucleotide of the invention.

In a further aspect, the present invention provides a host cell or extract thereof comprising a polynucleotide of the invention and/or a vector of the invention.

Examples of suitable host cells include, but are not limited to, a bacterial cell, a yeast cell or a plant cell. Preferably, the host cell is a bacterial cell. In one embodiment, the bacterial cell is an E. coli cell. In a further embodiment, the method is performed in a cell-free system either using an extract of a host cell of the invention and/or the vector of the invention in a cell-free system.

In another aspect, the present invention provides a method of producing a carbamate kinase, the method comprising cultivating a host cell of the invention or an extract thereof comprising the polynucleotide, or a vector of the invention, under conditions which allow expression of the polynucleotide encoding the carbamate kinase.

In an embodiment, the method produces at least 10 mg, more preferably at least 15 mg, of carbamate kinase from a gram of cells.

In a further aspect, provided is carbamoyl phosphate produced using a method of the invention.

Also provided is a compound produced using a method of the invention. Preferably, the compound is urea, citrulline, argininosuccinate, arginine, ornithine, or a pyrimidine.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. (A) Decomposition of the anion of carbamoyl phosphate. (B) Decomposition of the dianion of carbamoyl phosphate.

FIG. 2. HPLC analysis of multiple standard solutions containing AMP ADP and ATP at concentrations of: a) 0.2 mM; b) 0.4 mM; c) 0.6 mM; and d) 0.8 mM.

FIG. 3. pH dependence of Pfu CK (0.5 μM) catalytic production of ADP in NaHCO₃ (0.2 M), ATP (10 mM) and NH₄OH (▪=200 mM, ♦=20 mM, Δ=2 mM) at 40° C.

FIG. 4. Temperature dependence of Pfu CK catalytic production of ADP in NaHCO₃ (0.2 M), ATP (10 mM) and NH₄OH (20 mM) at pH 9.9.

FIG. 5. pH dependence of Pfu CK catalytic production of ADP after thirty minutes incubation in NaHCO₃, ATP (10 mM) and NH₄OH (200 mM and 20 mM) at 40° C.

FIG. 6. Comparison of integrations of carbon resonances observed in solutions of ammonia (2 M) and ¹³C-labelled sodium bicarbonate (0.2 M) in water, adjusted to pH 7.2, 8.4, 8.9, 9.4, 9.9, 10.4, 10.9 and 11.4.

FIG. 7. pH dependence of CKs catalytic production of ADP after thirty minutes incubation in NaHCO₃, ATP (10 mM) and NH₄OH (200 mM and 20 mM) at 40° C.

FIG. 8. Temperature dependence of CKs catalytic production of ADP in NaHCO₃ (0.2 M), ATP (10 mM) and NH₄OH (200 mM) at pH 9.9.

FIG. 9. Temperature dependence of CKs catalytic production of ADP in NaHCO₃ (0.2 M), ATP (10 mM) and NH₄OH (200 mM) at pH 9.9.

FIG. 10. Analysis of authentic urea by a) ¹H NMR and b) ¹³C NMR, dissolved in DMSO.

FIG. 11. Analysis of the urea product after incubation of carbamoyl phosphate (10 mg) in aqueous ammonia (2.5M) at 100° C. for 4 hours. a) ¹H NMR and b) ¹³C NMR, dissolved in DMSO.

FIG. 12. Analysis of the urea product after incubation of Pfu CK (0.5 μM) in a solution of sodium bicarbonate (0.2 M), ATP (10 mM) and aqueous ammonia (2.5M) at 100° C. for 4 hours. a) ¹H NMR and b) ¹³C NMR, dissolved in DMSO.

FIG. 13. Alignment of some carbamate kinases useful for the invention. Only amino acids which vary from P. fitriosus carbamate kinase are shown.

### KEY TO THE SEQUENCE LISTING

SEQ ID NO:1—Amino acid sequence of Pyrococcus furiosus carbamate kinase (NCBI Ref: NP_{—}578405.1).

SEQ ID NO:2—Amino acid sequence of Pyrococcus horikoshii carbamate kinase (NCBI Ref: NP_{—}143170).

SEQ ID NO:3—Amino acid sequence of Pyrococcus abyssi carbamate kinase (NCBI Ref: NP_{—}126565.1).

SEQ ID NO:4—Amino acid sequence of Thermococcus sp. carbamate kinase (NCBI Ref: ZP_{—}04879925.1).

SEQ ID NO:5—Amino acid sequence of Thermococcus gammatolerans carbamate kinase (NCBI Ref: YP_{—}002958486.1).

SEQ ID NO:6—Amino acid sequence of Thermococcus kodakarensis carbamate kinase (NCBI Ref: YP_{—}184571.1).

SEQ ID NO:7—Amino acid sequence of Thermococcus onnurineus carbamate kinase (NCBI Ref: YP_{—}002307889.1).

SEQ ID NO:8—Amino acid sequence of Thermococcus barophilus carbamate kinase (NCBI Ref: YP_{—}004071992.1).

SEQ ID NO:9—Amino acid sequence of Thermococcus sibiricus carbamate kinase (NCBI Ref: YP_{—}002995234.1).

SEQ ID NO:10—Codon optimized nucleotide sequence encoding Pyrococcus furiosus carbamate kinase.

SEQ ID NO:11—Nucleotide sequence encoding Pyrococcus furiosus carbamate kinase (NCBI Ref: NC_{—}003413).

SEQ ID NO:12—Codon optimized nucleotide sequence encoding Pyrococcus horikoshii carbamate kinase.

SEQ ID NO:13—Nucleotide sequence encoding Pyrococcus horikoshii carbamate kinase (NCBI Ref: NC_{—}000961).

SEQ ID NO:14—Codon optimized nucleotide sequence encoding Pyrococcus abyssi carbamate kinase.

SEQ ID NO:15—Nucleotide sequence encoding Pyrococcus abyssi carbamate kinase (NCBI Ref: NC_{—}000868).

SEQ ID NO:16—Codon optimized nucleotide sequence encoding Thermococcus sp. carbamate kinase.

SEQ ID NO:17—Nucleotide sequence encoding Thermococcus sp. carbamate kinase (reverse complement of NCBI Ref: NZ_DS999059).

SEQ ID NO:18—Codon optimized nucleotide sequence encoding Thermococcus gammatolerans carbamate kinase.

SEQ ID NO:19—Nucleotide sequence encoding Thermococcus gammatolerans carbamate kinase (NCBI Ref: NC_{—}012804).

SEQ ID NO:20—Codon optimized nucleotide sequence encoding Thermococcus kodakarensis carbamate kinase.

SEQ ID NO:21—Nucleotide sequence encoding Thermococcus kodakarensis carbamate kinase (NCBI Ref: NC_{—}006624).

SEQ ID NO:22—Codon optimized nucleotide sequence encoding Thermococcus onnurineus carbamate kinase.

SEQ ID NO:23—Nucleotide sequence encoding Thermococcus onnurineus carbamate kinase (NCBI Ref: NC_{—}011529).

SEQ ID NO:24—Codon optimized nucleotide sequence encoding Thermococcus barophilus carbamate kinase.

SEQ ID NO:25—Nucleotide sequence encoding Thermococcus barophilus carbamate kinase (NCBI Ref: NC_{—}014804).

SEQ ID NO:26—Codon optimized nucleotide sequence encoding Thermococcus sibiricus carbamate kinase.

SEQ ID NO:27—Nucleotide sequence encoding Thermococcus sibiricus carbamate kinase (NCBI Ref: NC_{—}012883).

SEQ ID NO:28—Amino acid sequence of Fervidobacterium nodosum carbamate kinase (NCBI Ref: A7HNY8).

SEQ ID NO:29—Amino acid sequence of Thermosipho melanesiensis carbamate kinase (NCBI Ref: A6LPA8).

SEQ ID NO:30—Amino acid sequence of Anaerobaculum hydrogeniformans carbamate kinase (NCBI Ref: D3L0Z7).

SEQ ID NO:31—Amino acid sequence of Aminobacterium colombiense carbamate kinase (NCBI Ref: D5ECR9).

SEQ ID NO:32—Amino acid sequence of Thermanaerovibrio acidaminovorans carbamate kinase (NCBI Ref: D1B8A3).

SEQ ID NO:33—Amino acid sequence of Halothermothrix orenii carbamate kinase (NCBI Ref: B8D2J8).

SEQ ID NO:34—Amino acid sequence of Kosmotoga olearia carbamate kinase (NCBI Ref: C5CE22).

SEQ ID NO:35—Amino acid sequence of Moorella thermoacetica carbamate kinase (NCBI Ref: Q2RGN0).

SEQ ID NO:36—Codon optimized nucleotide sequence encoding Fervidobacterium nodosum carbamate kinase.

SEQ ID NO:37—Codon optimized nucleotide sequence encoding Thermosipho melanesiensis carbamate kinase.

SEQ ID NO:38—Codon optimized nucleotide sequence encoding Anaerobaculum hydrogeniformans carbamate kinase.

SEQ ID NO:39—Codon optimized nucleotide sequence encoding Aminobacterium colombiense carbamate kinase.

SEQ ID NO:40—Codon optimized nucleotide sequence encoding Thermanaerovibrio acidaminovorans carbamate kinase.

SEQ ID NO:41—Codon optimized nucleotide sequence encoding Halothermothrix orenii carbamate kinase.

SEQ ID NO:42—Codon optimized nucleotide sequence encoding Kosmotoga olearia carbamate kinase.

SEQ ID NO:43—Codon optimized nucleotide sequence encoding Moorella thermoacetica carbamate kinase.

SEQ ID NO:44—Amino acid sequence of Enterococcus faecalis carbamate kinase (NCBI Ref: P0A2X7).

SEQ ID NO:45—Amino acid sequence of Clostridium tetani carbamate kinase (NCBI Ref: Q890 W1).

SEQ ID NO:46—Codon optimized nucleotide sequence encoding Enterococcus faecalis carbamate kinase.

SEQ ID NO:47—Codon optimized nucleotide sequence encoding Clostridium tetani carbamate kinase.

SEQ ID NO:48 and 49—Oligonucleotide primers.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, enzymology, urea production, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−20%, more preferably +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, a “thermophile” is an organism, preferably a bacteria, which can survive at temperatures of about 45° C. to about 70° C. As used herein, a “hyperthermophile” is an organism, preferably a bacteria, which can survive at temperatures of about 70° C. to about 120° C.

### Synthesis of Carbamoyl Phosphate

Carbamoyl phosphate is a key metabolite for nitrogen transfer in biological systems and is synthesised by carbamoyl phosphate synthetase (CPS) and carbamate kinase (CK) enzymes. Pyrococcus furiosus (Pfu) CK (EC6.3.4.16) (SEQ ID NO:1) was originally classified as a CPS until it was discovered that its true substrate was carbamate and that it used only mole of ATP. Unlike CPS, which enzymatically produces the carbamate it then turns over to carbamoyl phosphate, the Pfu CK turns over carbamate formed chemically from carbon dioxide and ammonia (Equation 1).

NH₃+[CO₂+H₂O [symbol] HCO₃⁻+H⁺] [symbol] NH₂CO₂⁻+H₃O⁺ (1)

The equilibrium formed with ammonia, carbon dioxide and carbamate is largely 20 determined by the ratio of CO₂ to NH₃ (Mani et al., 2006). When equal amounts of ammonia and CO₂ are in solution, ammonium bicarbonate is the primary product (Equation 2). If an excess of ammonia is available then the equilibrium favours the formation of ammonium carbamate (Equation 3).

$$\begin{matrix}{NH}_{3} + {CO}_{2} + {H}_{2} O {⇌}_{{K}_{eq} ( 293 ) = 1.02 \times {10}^{3}} {NH}_{4}^{+} + {HCO}_{3}^{-} & ( 2 ) \\ 2 {NH}_{3} + {CO}_{2} + {H}_{2} O {⇌}_{{K}_{eq} ( 293 ) = 3.63 \times {10}^{3}} {NH}_{2} {CO}_{2}^{-} + {NH}_{4}^{+} & ( 3 )\end{matrix}$$

The present inventors have developed a method where ammonia can be converted to carbamoyl phosphate in a single process. An advantage of this system is that it can be performed at both high pH and at low concentrations of ammonia. The high pH ensures that most of the ammonia is not in the form of ammonium, whereas the relatively low concentration of ammonia makes the method suitable for removing ammonia from sources such as biological and water waste products.

Enzyme pH-rate profiles provided in the Examples indicate rate maxima of carbamate kinase at approximately pH 9.9 in the presence of 2 mM, 20 mM and 200 mM ammonia. This is in contrast to results reported by Durbecq et al. (1997). It is also apparent at the ammonia concentrations studied, more neutral pH levels are actually detrimental to carbamoyl phosphate synthesis due to associated reductions in carbamate availability.

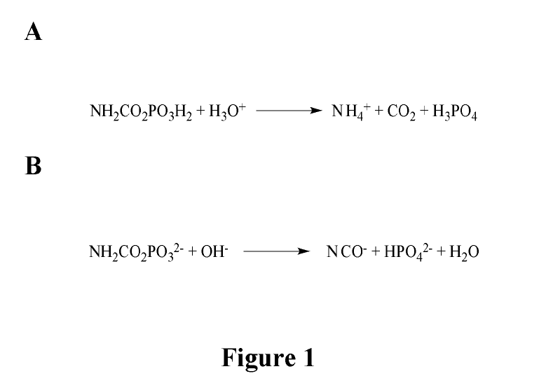

Product stability is also affected by pH. Carbamoyl phosphate is unstable in aqueous environments and readily decomposes (Wang et al., 2008). The path through which decomposition occurs is pH dependent. The anion, present from pH 2 to 4, decomposes to ammonia, carbonate and phosphate (FIG. 1A), whereas the dianion, present in alkaline conditions, decomposes to phosphate and cyanate (FIG. 1B). The path of decomposition is important for subsequent transformations as further nitrogen substitution is not possible with ammonia and carbonate but is known to occur readily with cyanate (Wen and Brooker, 1994). For example, the formation of urea does not occur when carbonate is treated with ammonia, however it does form when one or more of cyanate and cyanic acid is treated with ammonia and the production of urea increases with increased ammonia concentration.

Preferably, the ammonia concentration in the reaction to produce carbamoyl phosphate is at least about 1 mM. In an embodiment, the ammonia concentration is about 1 mM to about 5 M. In another embodiment, the ammonia concentration is about 2 mM to about 2 M. When high concentrations of ammonia are present, urea can be formed from carbamoyl phosphate through an intermediate as described herein.

In an embodiment, the ATP concentration is at least about 0.1 mM. In another embodiment, the ATP concentration is about 0.1 mM to about 100 mM. In a further embodiment, the ATP concentration is about 1 mM to about 20 mM. In yet another embodiment, the ATP concentration is about 10 mM.

In an embodiment, the bicarbonate is provided as sodium bicarbonate. In an embodiment, the bicarbonate concentration is at least about 10 mM. In another embodiment, the bicarbonate concentration is about 10 mM to about 1M. In a further embodiment, the bicarbonate concentration is about 100 mM to about 500 mM. In yet another embodiment, the bicarbonate concentration is about 200 mM.

Depending on the temperature a specific enzyme can tolerate, the reaction can be performed at a range of temperatures including about 10° C. to about 100° C. In an embodiment, the temperature is about 10° C. to about 80° C. However, in some circumstances it may be more economical and/or practical (such as when using ammonium/ammonia in waste water) to perform the reaction at a temperature lower than the optimal temperature of the enzyme, such as about 20° C. to about 30° C. For certain enzymes, such as those provided as SEQ ID NOs 1 to 9 at higher temperatures, such as about 90° C. to about 100° C., and in the presence of sufficient levels of ammonia, urea will be produced.

In another embodiment, the carbamate kinase maintains at least 30%, at least 40%, at least 50%, or at least 60% of its maximum activity at pH 10.5, 11 or 11.5. This can be determined, for instance, using the procedures described in Example 5 using an NH₄OH concentration of 20 mM or 200 mM.

The invention can be used to remove, or at least reduce the concentration of, ammonia from water, such as waste water, material. For instance, the waste can be derived from agricultural or industrial processes. In one embodiment, the waste material is in a liquid such as water from a dam or a stream. In another embodiment, the waste material is sewage, particularly comprising human and/or animal waste. In another embodiment, the waste material is a gas such as exhaust gas.

In an embodiment, the waste material is in a liquid which is clarified to remove suspended solids. The clarification may be carried out using conventional equipment such as a relief clarifier, a polishing filter, etc.

### Synthesis of Urea

Two different pathways have been proposed for the conversion of ammonium and cyanate (for example) into urea. The first is through an ionic mechanism NH₄⁺+NCO⁻→CO(NH₂)₂ and the other is a non-ionic mechanism with the reaction of ammonia and cyanic acid NH₃+HNCO→CO(NH₂)₂. The most convincing evidence of which is the actual mechanism was reported by Wen and Brooker (1994). They reported that the rate of reaction of cyanate to urea was directly proportional to the concentration of ammonium rather than ammonia which supports an ionic mechanism.

Carbamoyl phosphate produced using the methods of the invention can be used to synthesize urea. Ammonia is required for the reaction, which is generally performed at a high temperature of at least about 90° C.

In an embodiment, urea is produced in single vessel, preferably in a continuous system.

In an alternate embodiment, urea is produced in a number of stages. For instance, whilst CK is functional at temperatures above 90° C. (for example 90° C. to 100° C.), the production of carbamoyl phosphate is typically more efficient at lower temperatures (for example about 60° C.). In one example, carbamoyl phosphate is produced in accordance with the invention with the enzyme immobilized on a solid support. The carbamoyl phosphate produced is then separated from the solid support, for example by the solid support being in the form of a column and the carbamoyl phosphate being eluted from the column. The eluted carbamoyl phosphate can then be bound to a suitable resin followed by washing the resin in a solution comprising ammonia to convert the carbamoyl phosphate via an intermediate as defined herein into urea. This not only produces the urea but also liberates the molecule from the resin allowing the urea to be collected in the material eluted from the resin.

Resins suitable for the invention include mono- or di-anionic resins, such as those used for the removal of phosphate from wastewater and soil. Examples include KFR-3tT-40, SBS-3 and Amberlite IRA-400 (Rohm & Haas).

Preferably, the ammonia concentration in the reaction to produce urea is at least about 2.5M. In an embodiment, the ammonia concentration is about 2.5M to about 40M. In another embodiment, the ammonia concentration is about 2.5M to about 10M.

### Carbamate Kinases

As used herein, a “carbamate kinase” or “CK” is an enzyme capable of converting carbamate to carbamoyl phosphate. In the methods of the invention, ammonia and CO₂, or hydrated form thereof, are converted to carbamate in a chemical reaction, and the resulting carbamate and ATP are converted to carbamoyl phosphate in an enzyme-catalysed reaction by the carbamate kinase. A carbamate kinase used in the methods of the invention may or may not have some carbamoyl phosphate synthetase (CPS) activity, and thus be able to synthesises carbamoyl phosphate irreversibly from ammonia, bicarbonate and ATP in three steps. In a preferred embodiment, the carbamate kinase has no CPS activity.

The terms “polypeptide”, “protein” and “carbamate kinase” are generally used interchangeably and refer to a single polypeptide chain which may or may not be modified by addition of non-amino acid groups. It would be understood that such polypeptide chains may associate with other polypeptides or proteins or other molecules such as co-factors. The terms “proteins”, “polypeptides”, “carbamate kinase” as used herein also include variants, mutants, biologically active fragments, modifications, analogous and/or derivatives of the polypeptides described herein.

The % identity of a polypeptide is determined by GAP (Needleman and Wunsch, 1970) analysis (GCG program) with a gap creation penalty=5, and a gap extension penalty=0.3. The query sequence is at least 25 amino acids in length, and the GAP analysis aligns the two sequences over a region of at least 25 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the query sequence is at least 300 amino acids in length and the GAP analysis aligns the two sequences over a region of at least 300 amino acids. Even more preferably, the GAP analysis aligns the two sequences over their entire length.

As used herein a “biologically active fragment” is a portion of a polypeptide as described herein which maintains the ability to convert carbamate and ATP into carbamoyl phosphate. Biologically active fragments can be any size as long as they maintain the defined activity. Preferably, biologically active fragments are at least 200, more preferably at least 300, amino acids in length.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 55%, more preferably at least 60%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of a polypeptide described herein can be prepared by introducing appropriate nucleotide changes into a nucleic acid defined herein, or by in vitro synthesis of the desired polypeptide. Such mutants include, for example, deletions, insertions or substitutions of residues within the amino acid sequence. A combination of deletion, insertion and substitution can be made to arrive at the final construct, provided that the final polypeptide product possesses the desired characteristics.

Mutant (altered) polypeptides can be prepared using any technique known in the art. For example, a polynucleotide described herein can be subjected to in vitro mutagenesis. Such in vitro mutagenesis techniques may include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides described herein (for example two or more of SEQ ID NOs 10 to 27 or 36 to 43) are subjected to DNA shuffling techniques as broadly described by Harayama (1998). Products derived from mutated/altered DNA can readily be screened using techniques described herein to determine if they have carbamate kinase activity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. Sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1.

In a preferred embodiment a mutant/variant polypeptide has one or two or three or four conservative amino acid changes when compared to a naturally occurring polypeptide, or up to 10 or 15 or 20 amino acid changes relative to a reference sequence such as, for example, SEQ ID NOs: 1 to 9 or 28 to 35. Details of conservative amino acid changes are provided in Table 1. As the skilled person would be aware, such minor changes can reasonably be predicted not to alter the activity of the polypeptide when expressed in a recombinant cell.

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe; ala |

The crystal structure of the P. furiosus CK (SEQ ID NO:1), and structure/function relationships, have been described by Ramon-Maiques et al. (2000). Their studies discuss how the structure relates to its thermostability as well as how the structure of the active site relates to its function. They were able to conclude that the thermostability of the enzyme may result from the extension of the hydrophobic inter-subunit contacts and from the large number of exposed ion-pairs, and the slow rate at 37° C. is possibly a consequence of slow product dissociation. Ramon-Maiques et al. (2000) attributed the slow product dissociation to the strong binding of the purine ring which is “sandwiched between Met274 and TYR244” and well as the binding of the oxygen atoms of His268 and Ala241 forming hydrogen bonds with adenine NH₂ and NH of Ala241 makes another hydrogen bond at adenine N1. They state that these bonds should result in a high degree of specificity of the enzyme for adenine nucleotides. Furthermore, the carbamoyl moiety interacts with 10Gly-Gly-Asn and 52Gly-Asn-Gly, and the phosphoryl transfer involves three fully conserved lysine residues, Lys131, Lys215 and Lys277 (Ramon-Maiques et al., 2000), all of which are conserved in the enzymes tested in Example 5. In addition, Asp65 and Tyr71 defining 2-fold symmetry related interactions between alpha beta helices, whereas Gln94 participates in extending the network of hydrogen bonds. Asp65 is conserved in all of the Thermococci analysed. The charged residue is conserved throughout all of the carbamate kinases tested as glutamate or aspartate. Amino acid number 71 of P. furiosus CK is tyrosine or histidine in all of the Thermococci but is not present in the other carbamate kinases tested. Amino acid number 94 of P. furiosus CK is glutamine or glycine in the Thermococci, and an alanine or glutamine in the other carbamate kinases tested. As the skilled person would be aware, studies such as those by Ramon-Maiques et al. provide considerable guidance for the design of functional variants of naturally occurring CKs useful for the invention.

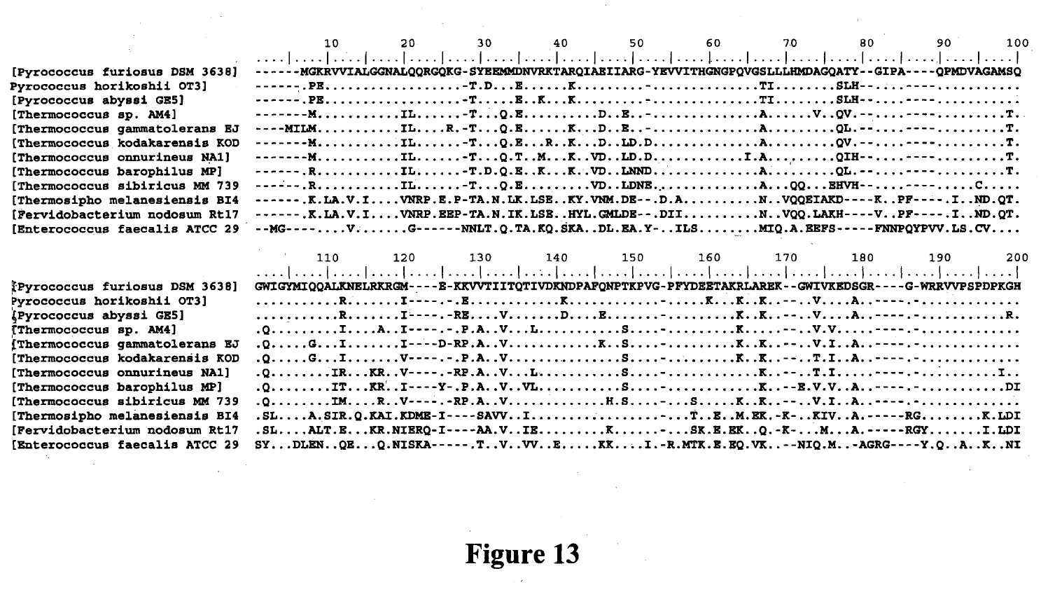

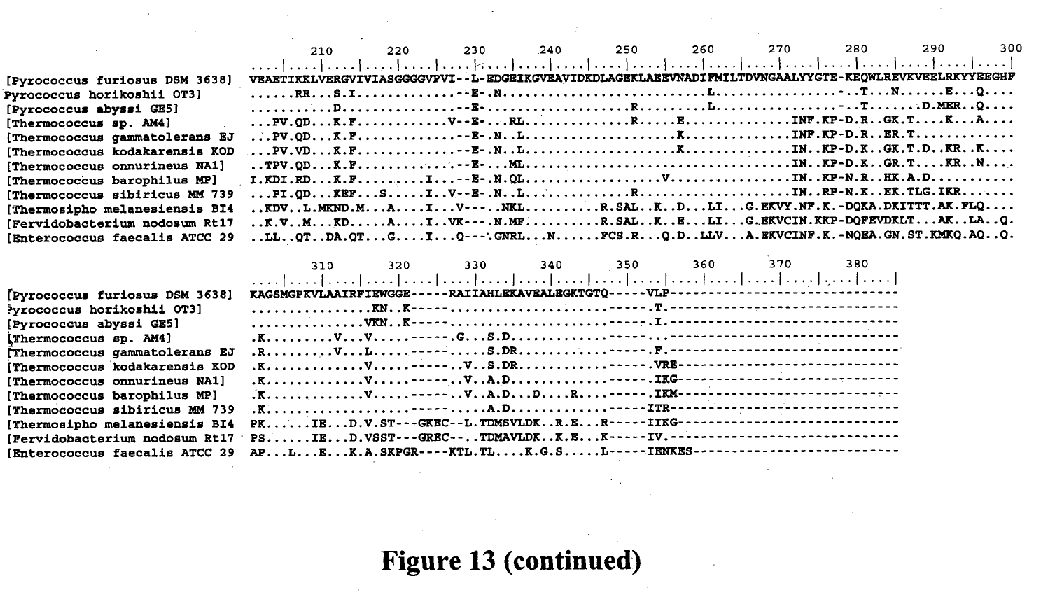

As the skilled person will appreciate, the polypeptides described herein (for example those with a sequence provided in two or more of SEQ ID NOs: 1 to 9 or 28 to 35) can be aligned to assist in the design of variant/mutant enzymes (see, for example, FIG. 13). Preferably, highly conserved amino acids are maintained, or possibly substituted in a conservative manner (see Table 1). In a further embodiment, if an amino acid of a protein is altered, it is substituted with an amino acid found in a corresponding position of another carbamate kinase such as one of those provided as SEQ ID NOs: 1 to 9 or 28 to 35.

Furthermore, if desired, unnatural amino acids or chemical amino acid analogues can be introduced as a substitution or addition into a polypeptide described herein. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are polypeptides which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide.

Polypeptides described herein can be produced in a variety of ways, including production and recovery of natural polypeptides, production and recovery of recombinant polypeptides, and chemical synthesis of the polypeptides. In one embodiment, the enzyme is produced by culturing a cell capable of expressing the polypeptide under conditions effective to produce the polypeptide, and recovering the polypeptide. A preferred cell to culture is a recombinant cell as defined herein. Effective culture conditions include, but are not limited to, effective media, bioreactor, temperature, pH and oxygen conditions that permit polypeptide production. An effective medium refers to any medium in which a cell is cultured to produce the polypeptide. Such medium typically comprises an aqueous medium having assimilable carbon, nitrogen and phosphate sources, and appropriate salts, minerals, metals and other nutrients, such as vitamins. Cells can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art. In an alternate embodiment, the polypeptides described herein can be produced in a cell free-system. Cell-free systems typically comprise a reaction mix comprising biological extracts and/or defined reagents. The reaction mix will comprise a template for production of the polypeptide, e.g. DNA, mRNA, etc.; amino acids, enzymes and other reagents that are necessary for the synthesis, e.g. ribosomes, tRNA, polymerases, transcriptional factors, etc. For example, the biological extract can be from an E. coli, Thermococcus sp. or Pyrococcus sp. cell producing the polypeptide. Such synthetic reaction systems are well-known in the art, and have been described in the literature. The cell free synthesis reaction may be performed as batch, continuous flow, or semi-continuous flow, as known in the art.

In an embodiment, the enzyme comprises a signal sequence which is capable of directing secretion of the polypeptide from a cell. A large number of such signal sequences have been isolated, which include N- and C-terminal signal sequences. Prokaryotic and eukaryotic N-terminal signal sequences are similar, and it has been shown that eukaryotic N-terminal signal sequences are capable of functioning as secretion sequences in bacteria. An example of such an N-terminal signal sequence is the bacterial β-lactamase signal sequence, which is a well-studied sequence, and has been widely used to facilitate the secretion of polypeptides into the external environment. An example of C-terminal signal sequences is the hemolysin A (hlyA) signal sequences of E. coli. Additional examples of signal sequences include, without limitation, aerolysin, alkaline phosphatase gene (phoA), chitinase, endochitinase, α-hemolysin, MIpB, pullulanase, Yops and a TAT signal peptide.

### Polynucleotides

By an “isolated polynucleotide”, including DNA, RNA, or a combination of these, single or double stranded, in the sense or antisense orientation or a combination of both, dsRNA or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. Furthermore, the term “polynucleotide” is used interchangeably herein with the term “nucleic acid”.

The term “exogenous” in the context of a polynucleotide refers to the polynucleotide when present in a cell, or in a cell-free expression system, in an altered amount compared to its native state. In one embodiment, the cell is a cell that does not naturally comprise the polynucleotide. However, the cell may be a cell which comprises a non-endogenous polynucleotide resulting in an altered, preferably increased, amount of production of the encoded polypeptide. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components.

Polynucleotides of the present invention may possess, when compared to molecules provided herewith, one or more mutations which are deletions, insertions, or substitutions of nucleotide residues. Mutants can be either naturally occurring (that is to say, isolated from a natural source) or synthetic (for example, by performing site-directed mutagenesis on the nucleic acid).

Usually, monomers of a polynucleotide are linked by phosphodiester bonds or analogs thereof. Analogs of phosphodiester linkages include: phosphorothioate, phosphorodithioate, phosphoroselenoate, phosphorodiselenoate, phosphoroanilothioate, phosphoranilidate and phosphoramidate.

### Recombinant Vectors

One embodiment of the present invention includes a recombinant vector, which comprises at least one isolated/exogenous polynucleotide of the invention inserted into any vector capable of delivering the polynucleotide molecule into a host cell. Recombinant vectors can also be used to produce a carbamate kinase useful for the invention, for example a recombinant vector comprising a sequence of nucleotide provided as any one of SEQ ID NOs: 10 to 27 or 36 to 43, or a sequence of nucleotide at least 50% identical to one or more thereof. Such a vector contains heterologous polynucleotide sequences, that is polynucleotide sequences that are not naturally found adjacent to polynucleotide molecules encoding a carbamate kinase and that preferably are derived from a species other than the species from which the polynucleotide molecule(s) are derived. The vector can be either RNA or DNA, either prokaryotic or eukaryotic, and typically is a transposon (such as described in U.S. Pat. No. 5,792,924), a virus or a plasmid.

One type of recombinant vector comprises the polynucleotide(s) operably linked to an expression vector. The phrase operably linked refers to insertion of a polynucleotide molecule into an expression vector in a manner such that the molecule is able to be expressed when transformed into a host cell. As used herein, an expression vector is a DNA or RNA vector that is capable of transforming a host cell and of effecting expression of a specified polynucleotide molecule. Preferably, the expression vector is also capable of replicating within the host cell. Expression vectors can be either prokaryotic or eukaryotic, and are typically viruses or plasmids. Expression vectors include any vectors that function (i.e., direct gene expression) in recombinant cells, including in bacterial, fungal, endoparasite, arthropod, animal, and plant cells. Vectors of the invention can also be used to produce the polypeptide in a cell-free expression system, such systems are well known in the art.

“Operably linked” as used herein refers to a functional relationship between two or more nucleic acid (e.g., DNA) segments. Typically, it refers to the functional relationship of transcriptional regulatory element to a transcribed sequence. For example, a promoter is operably linked to a coding sequence, such as a polynucleotide defined herein, if it stimulates or modulates the transcription of the coding sequence in an appropriate host cell and/or in a cell-free expression system. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

In particular, expression vectors contain regulatory sequences such as transcription control sequences, translation control sequences, origins of replication, and other regulatory sequences that are compatible with the recombinant cell and that control the expression of polynucleotide molecules. In particular, recombinant molecules include transcription control sequences. Transcription control sequences are sequences which control the initiation, elongation, and termination of transcription.

Particularly important transcription control sequences are those which control transcription initiation, such as promoter, enhancer, operator and repressor sequences. Suitable transcription control sequences include any transcription control sequence that can function in at least one of the recombinant cells described herein. A variety of such transcription control sequences are known to those skilled in the art. Preferred transcription control sequences include those which function in bacterial, yeast, arthropod, nematode, plant or animal cells, such as, but not limited to, tac, lac, trp, trc, oxy-pro, omp/lpp, rrnB, bacteriophage lambda, bacteriophage T7, T7lac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells.

### Host Cells

Another embodiment of the present invention includes a host cell, or the use of a host cell, transformed with one or more recombinant molecules described herein or progeny cells thereof. Transformation of a polynucleotide molecule into a cell can be accomplished by any method by which a polynucleotide molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed polynucleotide molecules can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide defined herein. Host cells either can be endogenously (i.e., naturally) capable of producing polypeptides described herein or can be capable of producing such polypeptides after being transformed with at least one polynucleotide molecule as described herein. Host cells can be any cell capable of producing at least one protein defined herein, and include bacterial, fungal (including yeast), parasite, nematode, arthropod, animal and plant cells. Examples of host cells include Salmonella, Escherichia, Bacillus, Listeria, Saccharomyces, Spodoptera, Mycobacteria, Trichoplusia, BHK (baby hamster kidney) cells, MDCK cells, CRFK cells, CV-1 cells, COS (e.g., COS-7) cells, and Vero cells. Further examples of host cells are E. coli, including E. coli K-12 derivatives; Salmonella typhi; Salmonella typhimurium, including attenuated strains; Spodoptera frugiperda; Trichoplusia ni; and non-tumorigenic mouse myoblast G8 cells (e.g., ATCC CRL 1246). Useful yeast cells include Pichia sp., Aspergillus sp. and Saccharomyces sp. Particularly preferred host cells are bacterial cells, yeast cells or plant cells.

Recombinant DNA technologies can be used to improve expression of a transformed polynucleotide molecule by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules include, but are not limited to, operatively linking polynucleotide molecules to high-copy number plasmids, integration of the polynucleotide molecule into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules to correspond to the codon usage of the host cell (see, for example, SEQ ID NOs:10, 12, 14, 16, 18, 20, 22, 24, 26 or 36 to 43), and the deletion of sequences that destabilize transcripts.

### Compositions

Compositions useful for the invention include excipients, also referred to herein as “acceptable carriers”. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal or o-cresol, formalin and benzyl alcohol. Excipients can also be used to increase the half-life of a composition, for example, but are not limited to, polymeric controlled release vehicles, biodegradable implants, liposomes, bacteria, viruses, other cells, oils, esters, and glycols.

In an embodiment, the carbamate kinase is immobilized on a solid support. This can enhance the production of carbamoyl phosphate, and/or increase the stability of the polypeptide. For example, the polypeptide can be immobilized on a polyurethane matrix (Gordon et al., 1999), or encapsulated in appropriate liposomes (Petrikovics et al., 2000a and b). The polypeptide can also be incorporated into a composition comprising a foam such as those used routinely in fire-fighting (LeJeune et al., 1998). As would be appreciated by the skilled addressee, the carbamate kinase can readily be used in a sponge or foam as disclosed in WO 00/64539. Other solid supports useful for the invention include resins with an acrylic type structure, with epoxy functional groups, such as Sepabeads EC-EP (Resindion srl—Mitsubishi Chemical Corporation) and Eupergit C (Rohm-Degussa), or with primary amino groups, such as Sepabeads EC-has and EC-EA (Resindion srl—Mitsubishi Chemical Corporation). In any case, the polypeptide is brought in contact with the solid support and immobilized through the high reactivity of the functional groups (epoxides) or activation of the support with a bifunctional agent, such as glutaraldehyde, so as to bind the enzyme to the matrix. Other supports suitable for the invention are polystyrene resins, macroreticular resins and resins with basic functional groups, such as Sepabeads EC-Q1A, the polypeptide is absorbed on the resin and then stabilized by cross-linking with a bifunctional agent (glutaraldehyde).

In one embodiment, the composition is in the form of a controlled release formulation that is capable of slowly releasing the composition into the environment (including soil and water samples). As used herein, a controlled release formulation comprises a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Preferred controlled release formulations are biodegradable (i.e., bioerodible).

The concentration of the enzyme will depend on, for example, the nature of the sample to be decontaminated, the concentration of ammonia in the sample, and the formulation of the composition. The effective concentration of the enzyme within the composition can readily be determined experimentally using a method of the invention.

### Uses

### Carbamoyl Phosphate

The transformation of carbamoyl phosphate chemically or biologically in situ, gives rise to valuable commodities. Carbamoyl phosphate can be used to produce urea as outlined herein.

Carbamoyl phosphate can be transformed into an array of carbamoyl derivatives as it will react with nucleophiles through an intermediate such as cyanate and/or cyanic acid. Alcohols will react with cyanate to form carbamates (Love and Kormendy, 1963). For example, the alcohol functional group from phenol will react with carbamoyl phosphate to form phenyl carbamate, which is a commonly used synthetic precursor to urea derivatives (Xiao et al., 1997). Similarly, carbamoyl phosphate can react with thiols resulting in the production of carbamothioates, which are widely used as herbicides (Wootton et al., 1993). Carbamoyl phosphate can also be used to introduce urea functionality in peptides.

The replacement of amide bonds with urea substituents has been of interest and these unnatural peptides have been studied as HIV protease inhibitors (Kempf et al., 1991), as well as β-turn protein mimics (Nowick et al., 1995).

Carbamoyl phosphate has been shown to have a prophylactic and possible therapeutic effect on dental caries. It has been discovered that carbamoyl phosphate and other carbamate compounds have a salutary effect on stabilization or growth of bone tissue and bone density (US 20030096741).

Carbamoyl phosphate can be an energy source for reactions (US 20020168706).

Carbamoyl phosphate, when reacted with aspartic acid, can be used to form uridine-5′-monophosphate (US 20020058244).

Pyrimidines, pyrimidine nucleosides, and pyrimidine nucleotides are synthesized from aspartic acid and carbamoyl phosphate (derived from glutamine and CO₂) by way of a multi-step pathway (see O'Donovan and Neuhard, 1970).

Citrulline, formed biochemically from carbamoyl phosphate in the urea cycle, is used as a pharmaceutical for the treatment of heart disease (Barr et al., 2007).

### Urea

Over 90% of the world's urea production is used as a nitrogen-release fertilizer. Urea has the highest nitrogen content of all solid nitrogenous fertilizers in common use. Many soil bacteria possess the enzyme, urease, which catalyzes the conversion of the urea molecule to two ammonia molecules and one carbon dioxide molecule, thus urea fertilizers are very rapidly transformed to the ammonium form in soils. Ammonia and nitrate are readily absorbed by plants, and are the dominant sources of nitrogen for plant growth. Urea is highly soluble in water and is, therefore, also very suitable for use in fertilizer solutions (in combination with ammonium nitrate), e.g., in ‘foliar feed’ fertilizers. For fertilizer use, granules are preferred over prills because of their narrower particle size distribution which is an advantage for mechanical application.

Urea is usually spread at rates of between 40 and 300 kg/ha but rates vary. Smaller applications incur lower losses due to leaching. During summer, urea is often spread just before, or during rain to minimize losses from volatilization (process wherein nitrogen is lost to the atmosphere as ammonia gas). Urea dissolves in water for application as a spray or through irrigation systems.

Urea absorbs moisture from the atmosphere and therefore is typically stored either in closed/sealed bags on pallets, or, if stored in bulk, under cover with a tarpaulin. As with most solid fertilizers, storage in a cool, dry, well-ventilated area is recommended.

Urea is a raw material for the manufacture of many important chemical compounds, such as some plastics (for example, urea-formaldehyde resins), some adhesives (for example, urea-formaldehyde or the urea-melamine-formaldehyde used in marine plywood), potassium cyanate (industrial feedstock), and urea nitrate (explosive).

In automobile systems urea is used in selective non catalytic reduction and selective catalytic reduction reactions to reduce the NOx pollutants in exhaust gases from combustion from diesel, dual fuel, and lean-burn natural gas engines. The BlueTec system, for example, injects water-based urea solution into the exhaust system. The ammonia produced by the hydrolysis of the urea reacts with the nitrogen oxide emissions and is converted into nitrogen and water within the catalytic converter.

Urea can serve as a hydrogen source for subsequent power generation in fuel cells. Urea present in urine/wastewater can be used directly (though bacteria normally quickly degrade urea.) Producing hydrogen by electrolysis of urea solution occurs at a lower voltage (0.37v) and thus consumes less energy than the electrolysis of water (1.2v).

With regard to medical uses, urea is used in topical dermatological products to promote rehydration of the skin. If covered by an occlusive dressing, 40% urea preparations may also be used for nonsurgical debridement of nails. Certain types of instant cold packs (or ice packs) contain water and separated urea crystals. Rupturing the internal water bag starts an endothermic reaction and allows the pack to be used to reduce swelling. Like saline, urea injection is used to perform abortions. Urea is the main component of an alternative medicinal treatment referred to as urine therapy. Urea labelled with carbon-14 or carbon-13 is used in the urea breath test, which is used to detect the presence of the bacteria Helicobacter pylori in the stomach and duodenum of humans, associated with peptic ulcers.

Other possible uses for urea produced using the methods of the invention include, but are not limited to, a stabilizer in nitrocellulose explosives, a component of animal feed, a non-corroding alternative to rock salt for road de-icing, resurfacing of snowboarding halfpipes and terrain parks, a flavour-enhancing additive for cigarettes, an ingredient in hair removers, a browning agent in factory-produced pretzels, an ingredient in some skin cream, moisturizers, and hair conditioners, a reactant in some ready-to-use cold compresses for first-aid use, a cloud seeding agent, a flame-proofing agent, an ingredient in tooth whitening products, an ingredient in dish soap, a yeast nutrient for fermentation of sugars into ethanol, a nutrient used by plankton in ocean nourishment experiments for geoengineering purposes, an additive to extend the working temperature and open time of hide glue, a solubility-enhancing and moisture-retaining additive to dye baths for textile dyeing or printing, and a protein denaturant.

### EXAMPLES

### Example 1

### Production of Pyrococcus furiosus Carbamate Kinase

The current literature method for expression of Pfu CK involves PCR amplification of the enzyme's genomic DNA (cpkA, Y09829.1) using synthetic oligonucleotide primers, designed to introduce an NcoI site at the initiator ATG and a BlpI site downstream of the stop codon (5′-GTGGTTTCCATGGGTAAGAGGGTAGTGATTGC-3′ (SEQ ID NO:48) and 5′-GCATTCGCTAAGCTGGGTCTTCTAAAGTTCCTCAGG-3 (SEQ ID NO:49)) (Dubecq et al., 1997). PCR products are then digested with NcoI and BlpI restriction enzymes, inserted into the corresponding sites of the plasmid pET-15b and the recombinant Pfu CK plasmid (pCPS184) is transformed into E. coli DH5a cells. An additional plasmid (pSJS1240) is also transformed to allow expression of the tRNA codons for arginine (AGA and AGG) and isoleucine (ATA) (these codons are rarely used in E. coli and occur frequently in the Pfu CK gene). The transformed E. coli DH5a cells are then grown overnight in 3 L of Luria-Bertani medium (supplemented with 0.1 mg/mL ampicillin and 0.05 mg/mL spectinomycin) at 37° C. in a shaking incubator, and after a 3 hour induction with 1 mM isopropyl β-D-thiogalactoside, cells are harvested by centrifugation. Approximately 10 g of cells can be obtained using this method of expression (Dubecq et al., 1997).

Pfu CK is then isolated from these cells at 0-4° C. using a series of purification processes. Firstly, the cells are suspended in 50 mM Tris-HCl, pH 7.5, lysed by sonication (sonic oscillator, 250 W, 10 kHz, 20 minutes) and centrifuged (80000×g, 30 minutes). Ammonium sulphate is then added to 40% saturation, and the solution is stirred for 30 minutes and centrifuged (12000×g, 20 minutes). Ammonium sulphate concentration is then increased to 80% saturation and the solution is again stirred for 30 minutes and centrifuged (12000×g, 20 minutes). The Pfu CK pellet obtained after this ammonium sulphate fractionation is suspended in 50 mM Tris-HCl, pH 7.2, dialysed in the same buffer and Pfu CK is isolated in a series of chromatographic purifications (DEAE sepharose, Blue Sepharose, DEAE Affi-gel Blue (Bio-Rad)). Using this method of expression and purification, 50 g of cells yields approximately 0.75 mg of protein (0.015 mg/g) (Uriate et al., 1999).

To address this low protein yield, Pfu CK was instead obtained using a redesigned enzyme expression and purification protocol. Firstly, the Pfu CK genomic DNA sequence was optimised for E. coli expression, making redundant the use of pSJS1240. The optimised cpkA gene was then inserted into the T7 promoter vector pETMCSIII between the NdeI and EcoRI restriction sites for subsequent expression with an N-terminal (His)₆ tag, and the recombinant plasmid was transformed into E. coli BL21 (DE3). The transformed cells were grown overnight in 100 mL of Luria-Bertani medium (supplemented with 0.1 mg/mL ampicillin) at 37° C. in a shaking incubator, and cells were harvested by centrifugation (4000×g, 5 minutes). Approximately 1 g of cells was obtained.

Isolation of Pfu CK was carried out at 0-4° C. Cells were suspended in 20 mM sodium phosphate, pH 7.4, supplemented with 500 mM NaCl and 20 mM imidazole, and lysed using a French press. Cell debris was then removed by centrifugation (12000×g, 30 minutes) and Pfu CK was isolated from the supernatant by metal-ion affinity chromatography (His GraviTrap (GE Healthcare)) eluting with 20 mM sodium phosphate, pH 7.4, supplemented with 500 mM NaCl and 500 mM imidazole. Approximately 16 mg Pfu CK was isolated from the initial 1 g cell pellet, representing a 1,000-fold improvement in enzyme yield.

### Example 2

### Production of Carbamoyl Phosphate

Previous analysis of catalytic turnover of ATP to ADP by Pfu CK has involved a coupled assay of pyruvate kinase and lactate dehydrogenase (Uriarte et al., 1999). In the presence of phophoenolpyruvate, pyruvate kinase converts ADP to ATP and produces pyruvate, which is then converted to lactate by lactate dehydrogenase, with oxidation of NADH. In the steady state, the UV monitored decrease in NADH concentration is then used as a measure of Pfu CK catalysed ADP production. Since this kinetic analysis is based on the detection of a secondary product, an alternate assay to more directly measure Pfu CK catalysed ADP production was designed. This assay is based on that designed by Buchan (2009) for the HPLC monitored activity of tRNA synthetases.

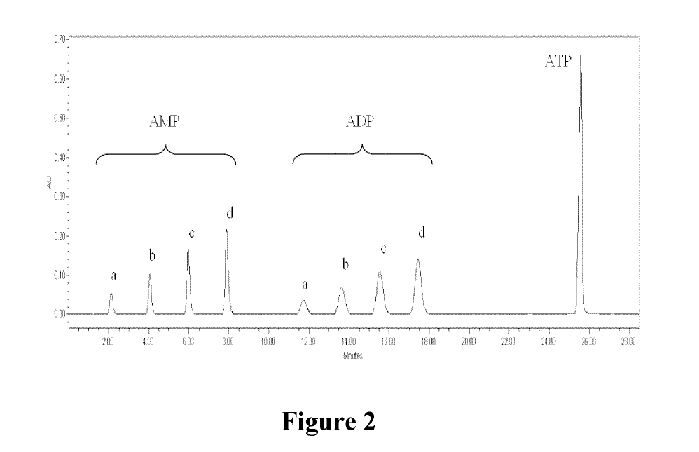

HPLC separation of AMP, ADP and ATP standard solutions was achieved using an Alltech Alltima HP C18 column eluting with a gradient of 60 mM ammonium dihydrogen phosphate and 5 mM tetrabutylammonium dihydrogen phosphate in water (solvent A) and 5 mM tetrabutylammonium phosphate in methanol (solvent B) according to the solvent system outlined in Table 2. The observed retention times for AMP, ADP and ATP standards were 7.9 minutes, 17.4 minutes and 25.6 minutes respectively. To expedite HPLC analyses, it was determined that 4 injections could be monitored per 27 minute analysis without co-elution of AMP or ADP (see FIG. 2).

Activity of Pfu CK for catalytic conversion of ATP to ADP was then monitored in various conditions. Assay solutions of 0.2 M sodium bicarbonate and 10 mM ATP were made containing 2 mM, 20 mM or 200 mM ammonia, and pH was adjusted to between pH 8.9 and 11.4 with 2 M NaOH. Assay temperature was maintained at 40° C. To allow buffered assays to be carried out at pH 8, 0.1 M Tris-HCl was added to assay mixtures and pH was adjusted using 5 M HCl (control assays confirmed that addition of Tris-HCl does not affect enzyme activity). Reactions were initiated by the addition of Pfu CK (0.5 μM final concentration) and ADP concentrations were monitored over 4 hours by quenching 20 μL reaction aliquots with 0.1% sodium dodecyl sulfate in water, followed by HPLC analysis as outlined in Table 2. ADP concentrations in reaction aliquots were determined using a standard calibration, and rates of change in ADP concentrations were corrected for background.

TABLE 2
HPLC solvent system used for the
separation of AMP, ADP and ATP.

| Time (minutes) | Solvent A (%) | Solvent B (%) |
|---|---|---|
| 0 | 87 | 13 |
| 18 | 87 | 13 |
| 19 | 70 | 30 |
| 23.2 | 70 | 30 |
| 24 | 87 | 13 |
| 27 | 87 | 13 |

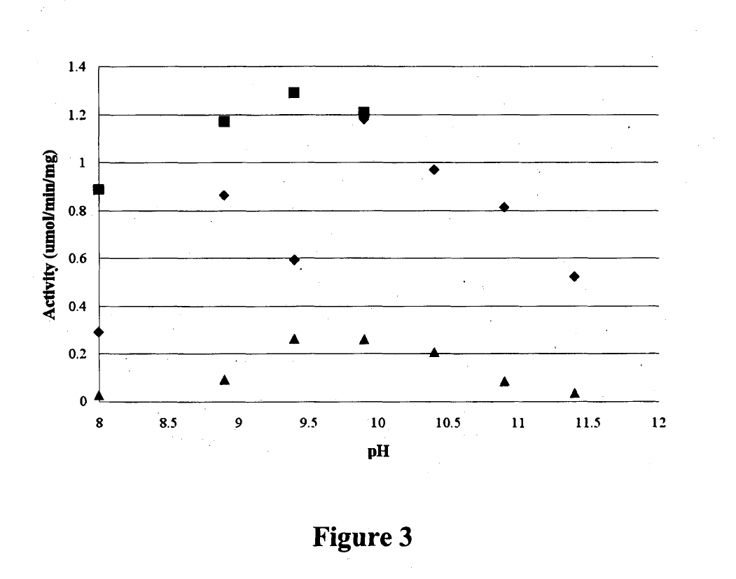

Shown in FIG. 3 are the pH-rate profiles of Pfu CK in the presence of 2 mM, 20 mM and 200 mM ammonia. Rate maxima are observed at approximately pH 9.9 at all three ammonia concentrations. At this pH, a ten-fold reduction in ammonia concentration from 200 mM to 20 mM has negligible effect on rate of ADP production, with both conditions allowing production of ADP at a rate of approximately 1.2 μmol/min/mg of enzyme. However, a further ten-fold reduction in ammonia concentration to 2 mM at pH 9.9 results in a 78% reduction in rate to 0.26 μmol/min/mg of enzyme. These results indicate that at pH 9.9, carbamate can be chemically synthesised at enzyme saturation concentrations upon mixing >20 mM ammonia with 0.2 M sodium bicarbonate. These results are in contrast to those of (Dubecq et al., 1997) which indicate that at 37° C. maximal activity was obtained between 7.8 and 8 and at 60° C. for a value near pH 7.6.

As can be seen in FIG. 3, a ten-fold reduction in ammonia concentration from 200 mM to 20 mM when below pH 9.9 has a negative effect on enzyme activity. This indicates that 20 mM ammonia is less able to produce carbamate at saturation concentrations at these conditions. This may be explained by a pH below the pKₐ of ammonia (9.25) reducing ammonia/ammonium ratios limiting concentrations of carbamate available to the enzyme. Increasing this ratio by increasing ammonia concentration ten-fold (200 mM) therefore increases carbamate concentrations closer to saturation levels. These trends are further evident using 2 mM ammonia, with decreases in rate at all pHs indicating further lowering of carbamate concentrations.

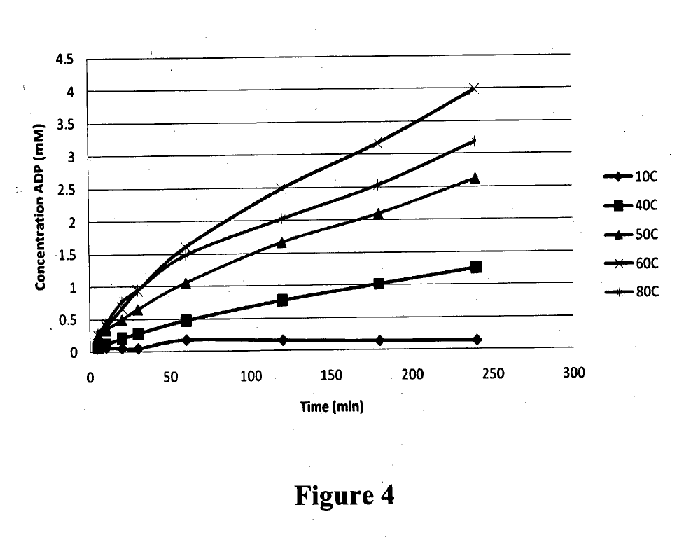

Further experiments investigating optimum temperature conditions were also carried out. This involved repeating assays of Pfu CK at pH 9.9 and in the presence of 20 mM ammonia as described above, whilst increasing assay temperatures from 40° C. to 60° C. and 80° C. Shown in FIG. 4 is the activity of Pfu CK for the production of ADP at these modified temperatures. An approximate three-fold increase in ADP 10 production was observed on increasing temperature from 40° C. to 60° C. However, a further increase in temperature to 80° C. did not result in a similar increase, with ADP production being approximately the same as observed at 60° C. These observations suggest an optimum temperature for enzyme activity of Pfu CK of between 60° C. and 80° C.

In summary, Pfu CK is capable of operating at variable temperatures, high pH and with low concentrations of ammonia and can therefore be used to remove toxic ammonia from wastewater to produce carbamoyl phosphate.

### Example 3

### Further Analysis of the Production of Carbamoyl Phosphate

Following obtaining the data presented in Example 2 the inventors conducted further analysis optimizing some of the parameters. The procedures used were as described in Example 1, but the cell debris was centrifuged at 20,000×g, 60 minutes and a 1,000-fold improvement in enzyme yield was obtained.

HPLC separation of AMP, ADP and ATP standard solutions was achieved using an Alltech Alltima HP C18 column eluting with a gradient of 60 mM ammonium dihydrogen phosphate and 5 mM tetrabutylammonium dihydrogen phosphate in water (solvent A) and 5 mM tetrabutylammonium phosphate in methanol (solvent B) according to the solvent system outlined in Table 3. The observed retention times for AMP, ADP and ATP standards were 8 minutes, 17.5 minutes and 25.5 minutes, respectively.

TABLE 3
HPLC solvent system used for the separation of
AMP, ADP and ATP. All gradients are linear.

| Time (minutes) | Solvent A (%) | Solvent B (%) |
|---|---|---|
| 0-18 | 87-70 | 13-30 |
| 18-19 | 70 | 30 |
| 19-23.2 | 70-87 | 30-13 |
| 23.2-27 | 87 | 13 |

Activity of Pfu CK for catalytic conversion of ATP to ADP was then monitored under various conditions. Assay solutions of 0.2 M sodium bicarbonate and 10 mM ATP were made containing 20 mM or 200 mM ammonia, and the pH was adjusted to between 8.9 and 11.4 with 5 M HCl or 2 M NaOH. The assay temperature was maintained at 40° C. Reactions were initiated by the addition of Pfu CK (0.5 μM final concentration) and ADP concentrations were monitored over 4 hours by quenching 20 μL reaction aliquots with 0.1% sodium dodecyl sulfate in water, followed by HPLC analysis as outlined in Table 3. ADP concentrations in reaction aliquots were determined using a standard calibration.

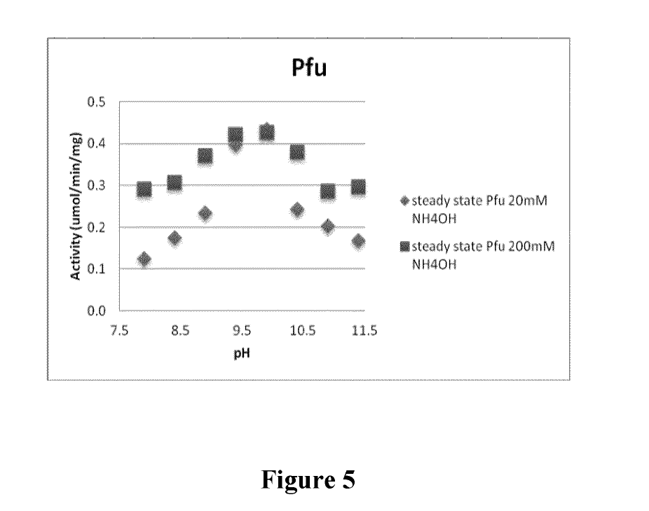

Shown in FIG. 5 are the pH-activity profiles of Pfu CK in the presence of 20 mM and 200 mM ammonia at a representative steady state from 10-30 minutes. Rate maxima are observed at approximately pH 9.9. More importantly, the activity is maintained at very high pH and only diminishes to one- to two-thirds of the maxima at pH 11.4. This is in contrast to mesophilic carbamate kinases, which have been reported to show a sharp decrease in activity above the pH maxima (Jones, 1962). These results show that Pfu CK has a rate maxima at 9.9 (40° C.); whereas the maxima was previously reported between 7.8 and 8 (Dubecq et al., 1997). In addition, this is the first report that Pfu CK is active at pH as high as 1 L4 at 40° C. It is possible that higher ammonia/ammonium ratios increases concentrations of carbamate available to the enzyme at high pH, and that this contributes to the surprising enzyme activity at high pH.

### Example 4

### Carbamate Speciation

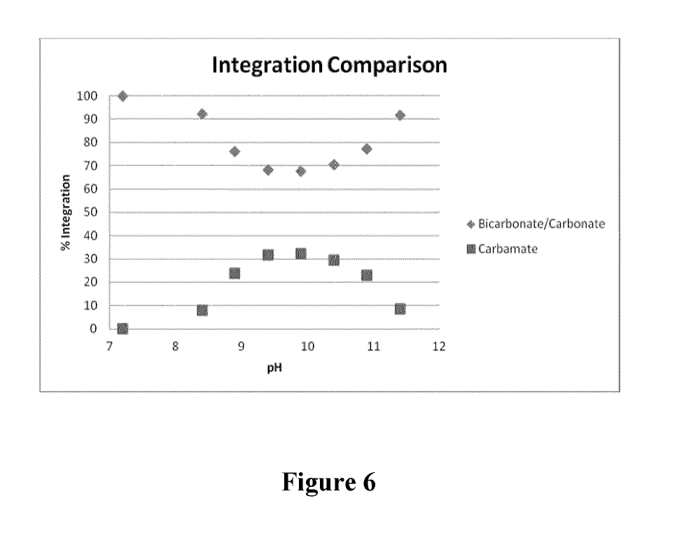

In order to determine if carbamate concentration relates to the Pfu CK pH profile, experiments were carried out monitoring carbamate concentration as a function of pH. Solutions of ammonia (2 M) and ¹³C-labelled sodium bicarbonate (0.2 M) in water were adjusted using either hydrochloric acid or sodium hydroxide to each of pH 7.2, 8.4, 8.9, 9.4, 9.9, 10.4, 10.9 and 11.4 and analysed by ¹³C NMR spectroscopy using a D₂O coaxial insert. At pH 7.2, a carbon resonance was observed at 159.5 ppm. This peak was assigned to the rapidly exchanging bicarbonate/carbonate pair. Increasing the pH to 8.4 caused this peak to shift to 160.0 ppm, with a second carbon resonance appearing at 164.9 ppm. This second peak was assigned to the carbamate formed through reaction of ammonia with bicarbonate (Mani et al., 2006). These peaks and their relative integrations were then monitored over the range of pH intervals from 7.2 to 11.4. Since these compounds contain only one carbon atom and are likely to display very similar relaxation times during NMR analysis (Mani et al., 2006), the integration of these peaks was used as a measure of their relative concentrations in solution. These integrations and chemical shifts are shown in Table 4. The trend in relative integrations is also shown in FIG. 6.

TABLE 4
Chemical shifts and relative integrations of carbon resonances
observed in solutions of ammonia (2M) and ¹³C-labelled
sodium bicarbonate (0.2M) in water, adjusted to pH 7.2,
8.4, 8.9, 9.4, 9.9, 10.4, 10.9 and 11.4.

| | HCO₃⁻/CO₃²⁻ | | Carbamate | |
|---|---|---|---|---|
| | | % Integration of | | % Integration of |
| | δ | HCO₃⁻/CO₃²⁻ to | δ | Carbamate to |
| pH | (ppm) | Carbamate | (ppm) | HCO₃⁻/CO₃²⁻ |
| 7.2 | 159.5 | 100 | — | 0 |
| 8.4 | 160.0 | 92 | 164.9 | 8 |
| 8.9 | 161.1 | 76 | 164.9 | 24 |
| 9.4 | 162.0 | 68 | 164.9 | 32 |
| 9.9 | 163.0 | 68 | 164.9 | 32 |
| 10.4 | 164.3 | 70 | 164.9 | 30 |
| 10.9 | 165.5 | 77 | 164.9 | 23 |
| 11.4 | 166.8 | 92 | 164.9 | 8 |

As is shown in Table 4, the carbon resonance assigned to the exchanging pair of bicarbonate/carbonate at pH 7.2 (159.5 ppm) moved to higher chemical shift with increasing pH. This effect was not observed with the carbon resonance assigned to carbamate, with a constant chemical shift of 164.9 ppm observed at all pH values. The relative integration of resonances for bicarbonate/carbonate and carbamate (FIG. 6) also shows that carbamate concentrations are highest at pH 9.9, with a 32% abundance of carbamate relative to bicarbonate/carbonate. This indicates that varying concentrations of the carbamate substrate may contribute to the activity profile of Pfu CK.

### Example 5

### Selection and Analysis of Additional Enzymes

In order to determine if the pH-activity profile of Pfu CK is peculiar to Pyrococcus furiosus a series of carbamate kinases from other organisms was selected for analysis and their activity compared to that of Pfu CK. The inventors tested carbamate kinases from other hyperthermophilic organisms as well as carbamate kinases from thermophilic and mesophilic species with similar structure to that of Pfu CK. Carbamate kinase structures were compared based on amino acid sequence homology relative to Pfu CK. The six enzymes chosen for further analysis are listed in Table 5.

TABLE 5
Carbamate kinase enzymes.

| | | Amino Acid |
|---|---|---|
| | | sequence homology |
| Enzyme | Abbreviation | relative to Pfu CK |
| 1. Thermococcus sibiricus | TS CK | 77.4% |
| 2. Thermococcus barophilus | TB CK | 76.8% |
| 3. Fervidobacterium nodosum | FN CK | 48.6% |
| 4. Thermosipho melanesiensis | TM CK | 48.1% |
| 5. Enterococcus faecalis | EF CK | 42.2% |
| 6. Clostrididium tetani | CT CK | 50.3% |

The carbamate kinase from Thermococcus sibiricus (TS CK, Table 5) was predicted based on sequence analysis Mardanov et al. (2009) but had not been isolated until now. Similarly, the complete genome sequence of Thermococcus barophilus (TB CK, Table 5) was reported in March 2011, however TB CK had not been isolated (Vannier et al., 2011). The genome sequence of Fervidobacterium nodosum was completed in 2007. This work was performed by the US Department of Energy's Office of Science, Biological and Environmental Research Program and by the University of California. The carbamate kinase from Fervidobacterium nodosum (FN CK, Table 5) had not been isolated. The carbamate kinase from Thermosipho melanesiensis (TM CK, Table 5) had again not been isolated but its genome was reported in 2009 (Zhaxybayeva et al., 2009). The carbamate kinase from Enterococcus faecalis (EF CK, Table 5) (formerly called Streptococcus faecalis) has been widely studied since 1964 (Kalman and Duffield, 1964). The carbamate kinase from Clostrididium tetani (CT CK, Table 5) had not been isolated previously. The genome sequence was completed in 2003 (Bruggemann et al., 2003). Table 6 provides a summary of the amino acid identity between the different enzymes tested.

TABLE 6
Amino acid sequence identity of carbamate kinases.

| | TB | TS | Pfu |
|---|---|---|---|
| CT | 52 | 53 | 53 |
| EF | 50 | 49 | 49 |
| FN | 54 | 55 | 52 |
| TM | 55 | 53 | 52 |
| Pfu | 77 | 78 | — |
| TS | 83 | — | 78 |
| TB | — | 83 | 77 |

With the exception of TB CK, the enzymes listed in Table 5 were expressed and purified using the optimised protocols as described above for Pfu CK. TB CK was successfully overexpressed by chemical induction using IPTG (isopropyl β-D-1-thiogalactopyranoside). For the IPTG induction, 10 mL preculture grown at 37° C. overnight was inoculated into 1 L LBA medium. The cells were grown until an OD of 0.55 was reached and then 1 mL IPTG (1 M, final 1 mM) was added. Cells induced with IPTG were grown overnight at 37° C. Cells were harvested by centrifugation, 5,000 rpm, 15 min, at 4° C. The purified yields of all additional enzymes are listed in Table 7. The yield of EF CK was 7 mg per 100 mL representing approximately 3-fold improvement from a previously reported expression (Marina et al., 1998). Furthermore, the protocol used involved a single purification step, easily completed in one day whereas the previous report involves multi-steps (including 2 columns and 2 precipitations) over a reported three day period (Marina et al., 1998).

TABLE 7
Purified yields of additional CK enzymes expressed.
Yields based on 100 ml liquid culture.

| Enzyme | Yield (mg) |
|---|---|
| TB CK | 3 |
| TS CK | 6 |
| FN CK | 4 |
| TM CK | 11 |
| EF CK | 7 |
| CT CK | 2 |

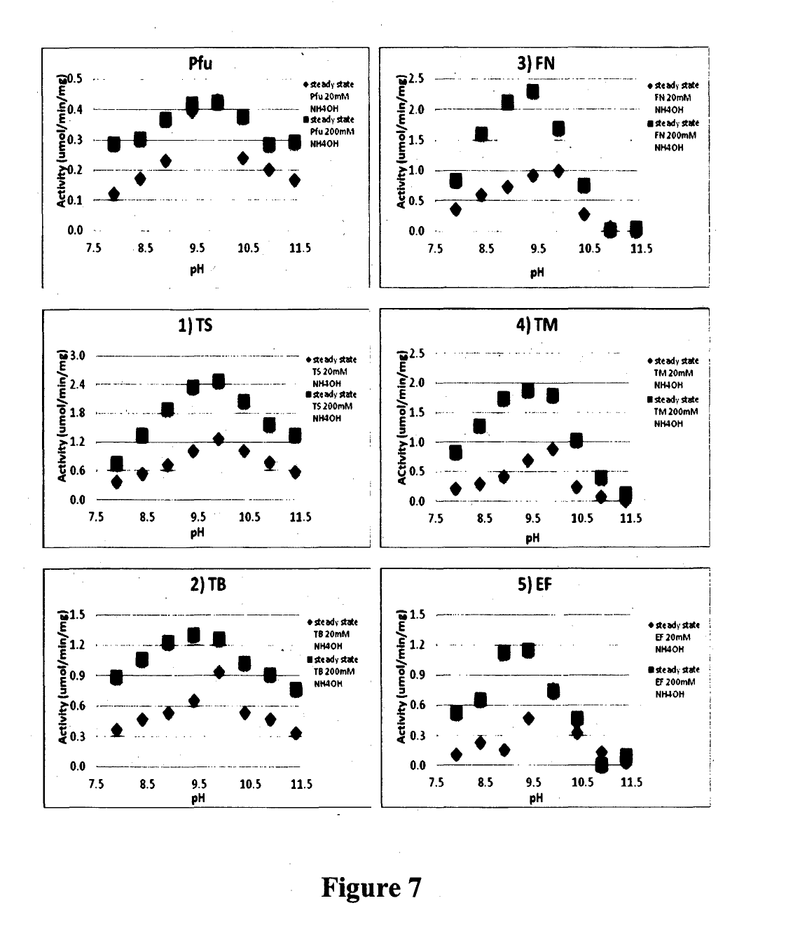

After expression and isolation, the CK enzymes where subjected to HPLC activity assays. Activity is based on a 20 minute assay after preincubation for 10 minutes and the pH profiles are shown in FIG. 7, for all but the mesophilic spore forming bacterial enzyme CT CK, which was not active under any of the conditions applied and was not studied further. Activity results show that the two hyperthermophilic enzymes (TS CK and TB CK, Table 5) with high sequence homology relative to Pfu CK exhibit pH activity profiles similar to that of Pfu CK (FIG. 7). As discussed above, Pfu CK maintains its activity at unusually high pH. Pfu CK, TS CK and TB CK have pH-rate maxima between 9.4-9.9 and retain a substantial proportion of this activity at pH 11.4. Based on these results it is expected that carbamate kinases with high sequence homology relative to CK Pfu will have similar pH-activity profiles.

The two carbamate kinase thermophiles (FN CK and TM CK) with less sequence homology relative to Pfu CK (Tables 5 and 6) had pH-activity profiles similar to those reported for mesophiles (Jones and Lipmann, 1960) where a sharp decrease in activity is observed from pH 9.4 to 10.4 (compared to little or no drop-off for Pfu CK, TS CK and TB CK) and they do not retain activity at pH 10.9-11.4 (FIG. 7).

As stated above, the mesophilic spore forming bacterial enzyme CT CK was not active under any of the conditions. The other mesophilic enzyme EF CK had a pH-activity profile similar to that of the thermophiles, FN CK and TM CK, with a sharp decrease in activity above pH 9.5 (FIG. 7).

Thus, the pH-activity profiles of the selected enzymes can be categorised by their amino acid sequence homology compared to Pfu CK.

### Example 6

### Temperature Profiles of Different Carbamate Kinases

In order to further investigate the correlation between structure and activity, temperature-activity experiments were carried out.

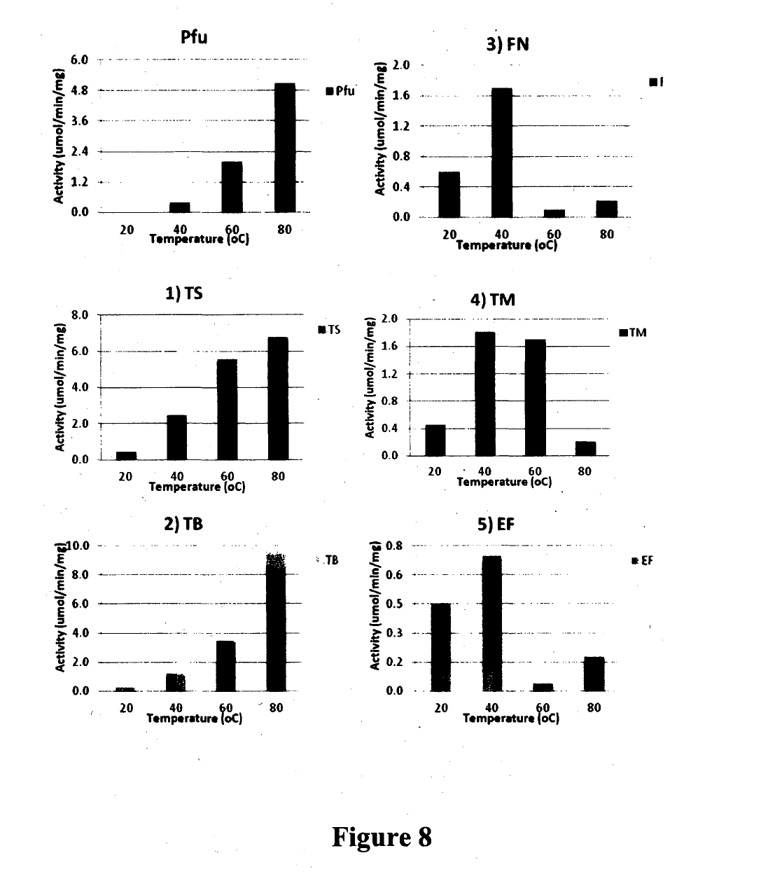

Assays at pH 9.9 and in the presence of 200 mM ammonia as described above were carried out, whilst maintaining assay temperatures of 20° C., 40° C., 60° C. and 80° C. FIG. 8 displays the steady state temperature profiles as a function of time at the designated temperatures. The carbamate kinases from the hyperthermophilic organisms and with high sequence homology relative to Pfu CK (TS CK and TB CK, Table 5) all have increasing activity with temperature with a maximum activity of 80° C. out of the four designated temperatures. At 20° C., enzymatic production of ADP is reduced to near background levels. Carbamate kinases from the thermophiles and with less sequence homology relative to Pfu (FN CK and TM CK, Table 5) are less active at the higher temperatures (FIG. 8). The FN CK was most active at 40° C. and the TM CK at 40-60° C. The active carbamate kinase from the mesophile (EF) was most active at 40° C. (FIG. 8) and showed little activity at 60° C. or 80° C. The ADP production at 80° C. in the EF CK profile in FIG. 8 is probably an artefact of background ATP hydrolysis.

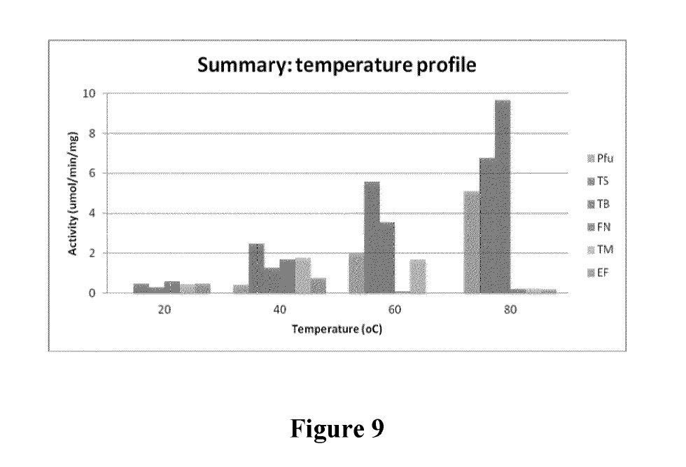

A temperature profile summary graph is shown in FIG. 9. At 80° C. Pfu CK as well as the carbamate kinases with high sequence homology relative to Pfu CK, have activities ranging from 5 to 9.5 mol/min/mg whilst the other carbamate kinases are not active at this temperature.

### Example 7

### Stability of Different Carbamate Kinases

Pfu CK and the similar sequence homology enzymes TS CK and TB CK are stable and unexpectedly function at high pH. In addition, they are stable and function, with increasing activity, at elevated temperatures and it follows that they are likely to retain function and structure on storage. These unusual characteristics are important attributes for commercial applications and the inventors therefore set-out to assess the stability of the carbamate kinases.

Preliminary stability assays were carried out for Pfu CK, TS CK, TB CK, FN CK, TM CK and EF CK. After incubation at 40° C. for 60 hours Pfu CK, TS CK and TB CK substantially maintained their activity whereas the others were inactive. Furthermore, Pfu CK retained its activity after being stored for a year at 4° C., demonstrating its potential for commercial applications.

### Example 8

### Production of Urea

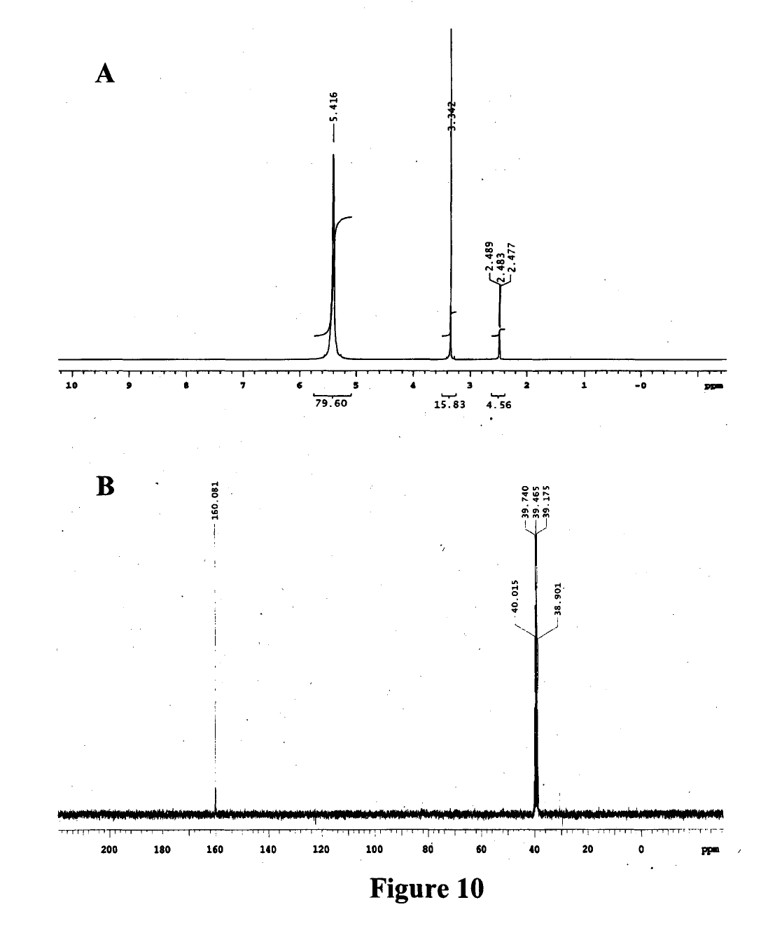

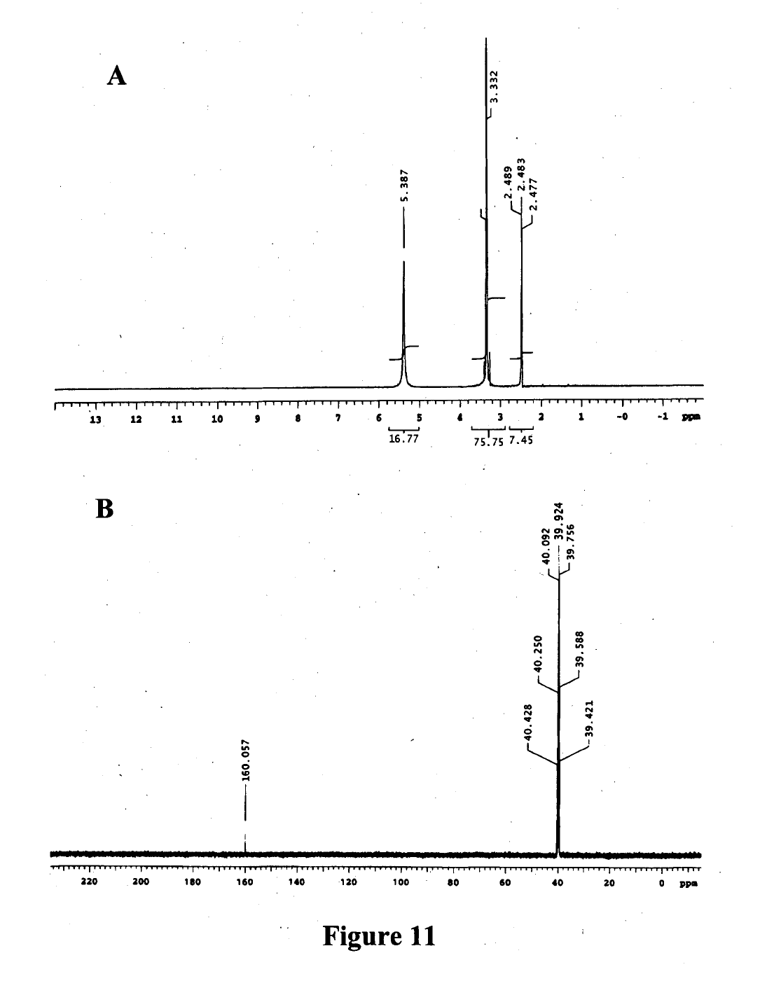

To determine the feasibility of in situ chemical conversion of biosynthesized carbamoyl phosphate to urea, model studies were carried out to determine the reactivity of carbamoyl phosphate in the presence of various concentrations of ammonia. Upon mixture of carbamoyl phosphate (50 mg) and liquid ammonia (10 mL) at −78° C., minimal dissolution was observed. It was thought that the low temperatures required to handle liquid ammonia were not amenable to carbamoyl phosphate solubility. Further experiments promoting carbamoyl phosphate solubility were therefore carried out. Carbamoyl phosphate (10 mg) was mixed with aqueous ammonia (1 mL, 14.8 M) and heated at 100° C. for 4 hours. Dissolution of carbamoyl phosphate was observed using these modified conditions. Upon drying the crude reaction product, analysis (TLC, ¹H/¹³C NMR, HPLC) with comparison to an authentic sample confirmed that the conversion of carbamoyl phosphate to urea had been achieved (see FIGS. 10 and 11 for spectral analyses). Additional experiments mixing carbamoyl phosphate with decreased concentrations of ammonia of 10 M, 5 M, and 2.5 M showed a negligible effect on urea production, as indicated by HPLC analysis. However, proceeding to ammonia concentrations below 2.5 M led to apparent reductions in urea concentrations observed.

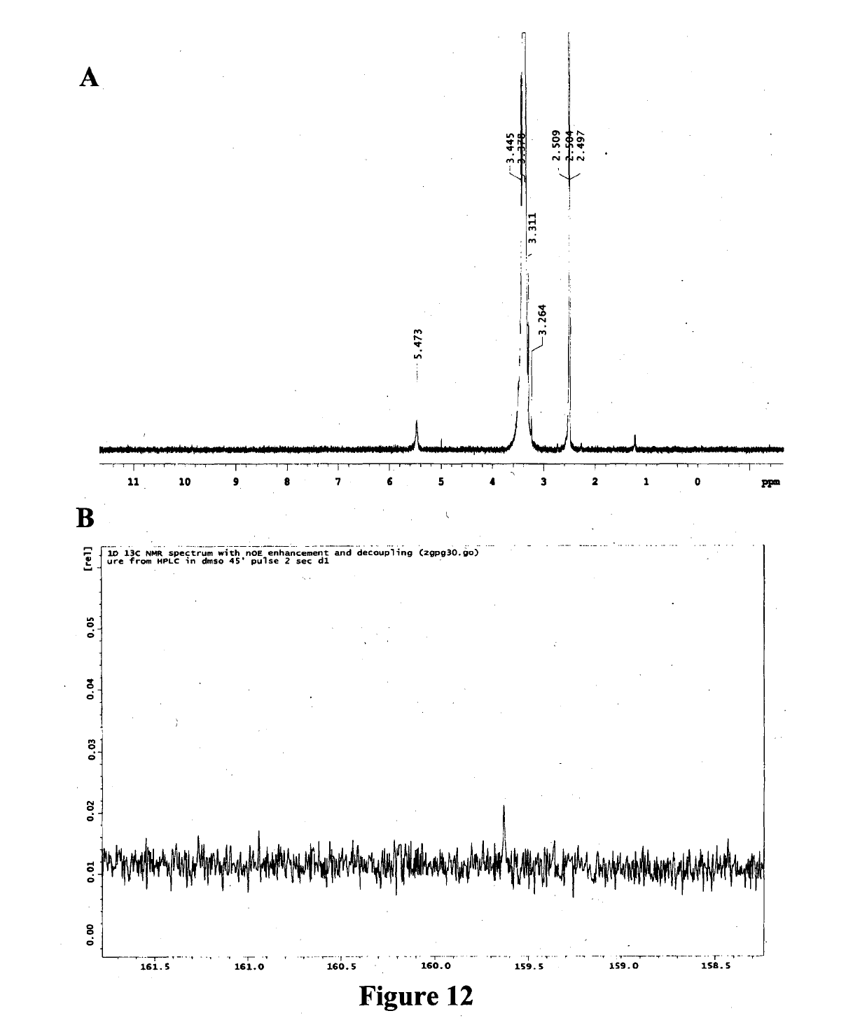

The conditions derived from these model studies were then applied to an assay of Pfu CK in an attempt to convert biosynthesised carbamoyl phosphate to urea. Pfu CK (0.5 μM final concentration) was added to a solution of 0.2 M sodium bicarbonate, 10 mM ATP and 2 M ammonia (pH 11) and the reaction was heated at 100° C. for 4 hours. The solution was then dried by nitrogen, and analysed by ¹H and ¹³C NMR (FIG. 12). These analyses confirmed that urea had been synthesised.

This application claims priority from U.S. 61/498,395 filed 17 Jun. 2011, the entire contents of which are incorporated herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Allen and Jones (1964) Biochemistry 3: 1238-1247.
Barr et al. (2007) J. Thorac. Cardiovasc. Surg. 134: 319-326.
Bruggemann et al. (2003) Proc. Natl. Acad. Sci. U.S.A. 100:1316-1321.
Buchan (2009) Study of Enzyme Catalysis of Cycloaddition Reactions. PhD Thesis, Australian National University: Canberra.
Durbecq et al. (1997) Proc. Natl. Acad. Sci. U.S.A. 94: 12803-12808.
Gordon et al. (1999) Chemical-Biological Interactions 14:463-470.
Harayama (1998) Trends Biotechnol. 16: 76-82.
Jones and Lipmann (1960) Proc. Natl. Acad. Sci. U.S.A. 46: 1194-1205.
Jones (1962) Carbamyl phosphate synthesis and utilization, in Methods in enzymology. Editor. Academic Press.
Kalman and Duffield (1964) Biochimica Et Biophysica Acta 92:498-512.
Kempf et al. (1991) Antimicrobial Agents and Chemotherapy 35: 2209-2214.
LeJuene et al. (1998) Nature 395:27-28.
Love and Kormendy (1963) J. Organic Chem. 28:3421-3428.
Mani et al. (2006) Green Chemistry 8: 995-1000.
Mardanov et al. (2009) Appl Environ Microbiol 75:4580-4588.
Marina et al. (1998) Eur J Biochem 253:280-291.
Needleman and Wunsch (1970) J. Mol. Biol. 48:443-453.
Nowick et al. (1995) J. Am. Chem. Soc. 117: 89-99.
O'Donovan and Neuhard (1970) Bacteriol. Rev. 34:278-343.
Petrikovics et al. (2000a) Toxicology Science 57: 16-21.
Petrikovics et al. (2000b) Drug Delivery 7: 83-89.
Ramon-Maiques et al. (2000) J Mol Biol 299:463-476.
Uriarte et al. (1999) J Biol Chem 274: 16295-16303.
Vannier et al. (2011) J Bacteriol 193:1481-1482.
Wang et al. (2008) Proc. Natl. Acad. Sci. U.S.A. 105: 16918-16923.
Wen and Brooker (1994) Canadian J Chemistry-Revue Canadienne De Chimie 72: 1099-1106.
Wootton et al. (1993) Bull. Environ. Contam. Toxicol. 50: 49-56.
Xiao et al. (1997) J. Organic Chem. 62: 6968-6973.
Zhaxybayeva et al. (2009) Proc. Natl. Acad. Sci. U.S.A. 106:5865-5870.

1. A method of producing carbamoyl phosphate, the method comprising reacting ammonia, ATP, bicarbonate and CO₂, or a hydrated form thereof, in a composition in the presence of a carbamate kinase, wherein the ammonia and CO₂, or hydrated form thereof, are converted to carbamate in a chemical reaction and the carbamate and ATP are converted to carbamoyl phosphate in an enzyme-catalysed reaction by the carbamate kinase, and wherein the pH of the composition is about 8 to about 12.

2. The method of claim 1, wherein the pH is about 9 to about 11, about 9.25 to about 11.25, about 10.25 to about 11.25, or about 10.5 to about 11.5.

3. The method of claim 2, wherein the carbamate kinase is derived from a hyperthermophile bacteria, or is a biologically active mutant thereof.

4. The method of claim 1, wherein the carbamate kinase comprises
a) an amino acid sequence provided as any one of SEQ ID NOs:1 to 9,
b) an amino acid sequence which is at least 50% identical to any one or more of SEQ ID NOs:1 to 9, and/or
c) a biologically active fragment of a) or b).

5. (canceled)

6. The method of claim 2, wherein the temperature is about 10° C. to about 80° C.

7. The method of claim 2, wherein
i) at pH 11 0.5 μM of carbamate kinase produces at least 0.5 μmol/min/mg ADP after thirty minutes incubation in NaHCO₃ (0.2 M), ATP (10 mM) and 20 mM NH₄OH at 40° C., or
ii) at pH 11.5 0.5 μM of carbamate kinase produces at least 0.25 μmol/min/mg ADP after thirty minutes incubation in NaHCO₃ (0.2 M), ATP (10 mM) and 20 mM NH₄OH at 40° C.

8. (canceled)

9. The method of claim 1, wherein the pH is about 9 to about 10.5, or about 9.5 to about 10.5.

10. The method of claim 9, wherein the carbamate kinase is derived from a thermophile bacteria, or is a biologically active mutant thereof.

11. The method of claim 9, wherein the carbamate kinase comprises
a) an amino acid sequence provided as any one of SEQ ID NOs:28 to 35,
b) an amino acid sequence which is at least 50% identical to any one or more of SEQ ID NOs:28 to 35, and/or
c) a biologically active fragment of a) or b).

12. The method of claim 9, wherein
i) at pH 10.5 0.5 μM of carbamate kinase produces at least 0.6 mmol/min/mg ADP after thirty minutes incubation in NaHCO₃ (0.2 M), ATP (10 mM) and 200 mM NH₄OH at 40° C., and/or
ii) temperature is about 10° C. to about 60° C.

13. (canceled)

14. The method of claim 1, wherein one or more of the following apply:
i) the temperature is about 20° C. to about 30° C.,
ii) the carbamate kinase maintains at least about 50%, at least about 60%, at least about 70%, or at least about 80% of its activity after storage for 1 year at 4° C. and/or storage for 60 hours at 40° C.,
iii) the pressure is about 0 to about 10 atm,
iv) which is performed in a continuous system,
v) the carbamate kinase is immobilized on a solid support,
vi) the source of the ammonia is waste material, and
vii) which further produces one or both of cyanate and cyanic acid through the decomposition of at least some of the carbamoyl phosphate.

15.-20. (canceled)

21. A method of producing a compound from carbamoyl phosphate, the method comprising
i) performing the method of claim 1 to produce carbamoyl phosphate, and
ii) performing one or more further reactions to produce the compound.

22. The method of claim 21, wherein the compound is urea and step ii) comprises reacting the carbamoyl phosphate produced from step i) with ammonia to produce urea via an intermediate, which is one or both of cyanate and cyanic acid.

23. The method of claim 22, wherein at least step ii) is performed at a temperature of at least about 90° C., or about 90° C. to about 100° C.

24.-25. (canceled)

26. The method of claim 21, wherein the compound is an intermediate of the urea cycle selected from citrulline, argininosuccinate, arginine, ornithine, and a combination of two or more thereof.

27.-29. (canceled)

30. The method of claim 21, wherein the method is performed in a single vessel.

31. A method of reducing the concentration of ammonia in a waste material, the method comprising performing a method of claim 1.

32. (canceled)

33. An isolated and/or exogenous polynucleotide encoding a carbamate kinase, wherein the polynucleotide comprises a sequence of nucleotides provided as any one of SEQ ID NOs:10, 12, 14, 16, 18, 20, 22, 24, 26 or 36 to 43.

34.-38. (canceled)

39. Carbamoyl phosphate produced using the method of claim 1.

40. A compound produced using the method of claim 21.

41. (canceled)
